(19) United States
(12) Patent Application Publication
(10) Pub. No.: US 2011/0130711 A1
(43) Pub. Date: Jun. 2, 2011

(54) HAIR GROWTH TREATMENT

(75) Inventors: Seth M. LEDERMAN, New York, NY (US); Scott C. KELLOGG, Mattapoisett, MA (US); Shikha P. BARMAN, Bedford, MA (US)

(73) Assignee: FOLLICA, INC., WALTHAM, MA (US)

(21) Appl. No.: 12/950,042

(22) Filed: Nov. 19, 2010

Related U.S. Application Data
(60) Provisional application No. 61/262,840, filed on Nov. 19, 2009.

(51) Int. Cl. A61M 37/00; A61B 17/3209; A61B 18/20

(52) U.S. Cl. 604/22; 606/167; 606/9

(57) ABSTRACT

The present disclosure provides methods and systems for increasing hair growth in subjects in need thereof due to male- or female-pattern hair loss, pathological hair loss, or hair loss after injury. By any use of any appropriate mechanical, electromagnetic, or chemical means, the present disclosure pertains to the segmentation of hair follicles into two or more disunited subunits in order to form new follicles from the respective subunits. In contrast with known techniques for bisecting hair follicles and as described more fully herein, the present methods and systems permit the bisection of single follicles or populations of follicles with a high degree of efficiency, and thereby represent an effective treatment option for those in need of increased hair growth.

### CROSS-REFERENCE TO RELATED APPLICATION

The present application claims priority to U.S. Provisional App. No. 61/262,840, filed Nov. 19, 2009, the entire contents of which are incorporated herein by reference.

### TECHNICAL FIELD

The present invention pertains to the injury to a body surface for promoting hair growth pursuant to a cosmetic or other medical treatment.

### BACKGROUND

The promotion of hair growth is desirable in the treatment of common baldness, skin injury (e.g., to reduce the appearance of scarring, aid in the healing of wounds, and improve skin rejuvenation), as well as less common conditions that are characterized by hair loss, such as discoid lupus, erythematosis, congenital hypotrichosis, lichen planopilaris, and other scarring alopecias, among other conditions. New follicles are either from new cells or from divisions of existing follicles.

One approach for promoting new hair growth is the induction of follicular neogenesis. Follicular neogenesis is the generation of new hair follicles after birth. Human beings are born with a full complement of hair follicles, which can change in size and growth characteristics (as in early baldness) or can ultimately degenerate and disappear (as in the late stages of baldness or in permanent scarring or cicatricial alopecias). In a mouse study, it was demonstrated that physically disrupting the skin and existing follicles, in a defined fashion, can lead to follicle neogenesis (Ito et al., 2007, Nature 447:316-321). Despite earlier suggestions of the regenerative capacity of the adult mammalian skin to recreate the embryonic follicle, follicle neogenesis was never proven because of the lack of understanding of the fundamental biology of the follicle (see Argyris et al., 1959, Dev. Biol. 1: 269-80; Miller, 1973, J. Invest. Dermatol. 58:1-9; and Kligman, 1959, Ann NY Acad Sci 83: 507-511). More recently, a series of murine experiments definitively showed that hair follicle-derived epithelial stem cell progenitors migrate out of the follicle and contribute to the re-epithelialization of injured skin (see Morris et al., 2004, Nature Biotechnology 22:411-417; Ito et al., 2004, Differentiation 72:548-57; and Ito et al., 2005, Nature Medicine 11:1351-1354).

Hair transplantation is another approach for providing hair on hair-deficient patches of skin, and involves the extraction of hair follicles from a donor location and implanting the donor follicles into a recipient site in need of hair growth. The transplanted hair follicles typically include some surrounding epidermis and dermis from the donor site. Transplants are performed on an outpatient basis under mild sedation, topical anesthesia, or both. The process for transplanting hair follicles is costly and time consuming, results vary widely from patient to patient, and side effects including shock loss and the appearance of patchiness have been reported on a regular basis.

Chemical treatments for promoting hair growth involve the use of drugs for the treatment of certain MPHL. These include, for example, minoxidil (an antihypertensive drug that opens the K+ channel); and antiandrogens such as finasteride, dutasteride or ketoconazole. While these types of treatments are reasonably effective in preventing or delaying MPHL, they are less effective in stimulating the growth of significant terminal hair in scalp of MPHL after baldness has been present for 6 months or more. Consequently, patients with advanced MPHL may express dissatisfaction with even statistically significant, but cosmetically insignificant increase in hair counts and such frustration may contribute to poor compliance and further unsatisfactory outcomes.

A device that uses low level light energy directly on the scalp (the HairMax® LaserComb®) to encourage hair growth has received FDA clearance as a 510K device. Although the device is advertised as a “laser,” it operates by applying low level monochromatic light energy directly to the scalp, which is thought to stimulate hair growth through “photobiostimulation” of hair follicles. Various types of devices operating on similar principles were referenced as the predicate for HairMax® (see Lolis et al., 2006, J. Cosmetic Dermatol. 5:274-276; Leavitt et al., 2009, Clin. Drug. Invest. 29:283-292).

There remains a need for additional methods and systems for providing the required conditions for promoting hair growth. The market demand for procedures would be generated by large numbers of individuals that are presently in need of hair augmentation due to male- or female-pattern hair loss, pathological hair loss, or hair loss after injury.

### SUMMARY

Conventional methods for increasing hair growth, including inducing follicular neogenesis, hair transplantation, or the use of low-level light energy, may provide a measure of success for some patients, but because no technique exists to satisfy every subject in need, there remains an ongoing search for new methods and systems. The present disclosure provides methods and systems that increase the number of hair-producing follicles on a body surface by two-fold or more, representing a valuable treatment for subjects suffering from male- or female-pattern hair loss, pathological hair loss, or hair loss after injury. The present methods and systems are able to maximize hair growth in and near areas where follicles exist but are too few in number to provide hair at a desired density, including the scalp, the face, and the margins of wounds and scars. These and other advantages will become readily apparent throughout the present disclosure.

In one aspect, methods for stimulating hair growth at a body surface are provided comprising: (a) identifying a first hair follicle on the body surface; (b) segmenting the first hair follicle into at least two disunited subunits in response to the identification; (c) optionally applying a composition to the site of the first hair follicle; and (d) identifying a further hair follicle; segmenting the further hair follicle into at least two disunited subunits; and optionally applying the same or a different composition to the site of the further hair follicle, or, (e) segmenting each of one or more further hair follicles into at least two disunited subunits contemporaneously with step (b); and optionally applying the same or a different composition to the site of the further hair follicle contemporaneously with step (c).

In another aspect, systems for stimulating hair growth at a body surface are provided comprising: an incision unit that is configured for applying a first incisor at an oblique angle relative to the body surface at the location of the first hair follicle for segmenting the first hair follicle into at least two disunited subunits.

### BRIEF DESCRIPTION OF THE DRAWINGS

FIG. 1 depicts how a fractional laser pattern may be adjusted in order to avoid an impediment.

FIG. 2 shows how an incisor may be applied at an oblique angle relative to a body surface in order to segment a hair follicle.

FIG. 3 illustrates the use of a fractional laser to form a hole in human skin, after which the hole is filled with a highly viscous drug-containing gel via an ink-jet precision fill device; body heat or other external factors then crosslink the gel into a stable drug-releasing matrix.

FIG. 4 depicts how the segmentation of a hair follicle at the margin of scar tissue can be used to generate new hair follicles for producing hair that grows into the scar tissue, thereby providing beneficial cosmetic results.

FIG. 5 shows exemplary incision units for applying a laser incisor to a body surface.

FIG. 6 depicts a novel design for the “cage” of a fractional laser having a substantially rhombohedron-shaped configuration in order to permit the delivery of laser beams at an angle that is not perpendicular to the body surface.

FIG. 7 depicts incision units comprising a row and incision units comprising an array.

FIG. 8 provides a trigonometric description of the laser angle φ, and the optimal length of injury (lᵢ) and the length of injury depth (l_{d})

FIG. 9 depicts how translation of an incisor may be used to form a “slice” injury in a body surface.

FIG. 10 shows a component of the present invention that features an integrated head design.

FIG. 11 illustrates an embodiment whereby an incision unit having a “row” configuration is translated relative to a body surface in a direction Z that is substantially transverse.

### DETAILED DESCRIPTION OF ILLUSTRATIVE EMBODIMENTS

The present inventions may be understood more readily by reference to the following detailed description taken in connection with the accompanying figures and examples, which form a part of this disclosure. It is to be understood that these inventions are not limited to the specific products, methods, conditions or parameters described and/or shown herein, and that the terminology used herein is for the purpose of describing particular embodiments by way of example only and is not intended to be limiting of the claimed inventions.

In the present disclosure the singular forms “a,” “an,” and “the” include the plural reference, and reference to a particular numerical value includes at least that particular value, unless the context clearly indicates otherwise. Thus, for example, a reference to “a composition” is a reference to one or more of such compositions and equivalents thereof known to those skilled in the art, and so forth. When values are expressed as approximations, by use of the antecedent “about,” it will be understood that the particular value forms another embodiment. As used herein, “about X” (where X is a numerical value) preferably refers to ±10% of the recited value, inclusive. For example, the phrase “about 8” preferably refers to a value of 7.2 to 8.8, inclusive; as another example, the phrase “about 8%” preferably (but not always) refers to a value of 7.2% to 8.8%, inclusive. Where present, all ranges are inclusive and combinable. For example, when a range of “1 to 5” is recited, the recited range should be construed as including ranges “1 to 4”, “1 to 3”, “1-2”, “1-2 & 4-5”, “1-3 & 5”, “2-5”, and the like. In addition, when a list of alternatives is positively provided, such listing can be interpreted to mean that any of the alternatives may be excluded, e.g., by a negative limitation in the claims. For example, when a range of “1 to 5” is recited, the recited range may be construed as including situations whereby any of 1, 2, 3, 4, or 5 are negatively excluded; thus, a recitation of “1 to 5” may be construed as “1 and 3-5, but not 2”, or simply “wherein 2 is not included.” It is intended that any component, element, attribute, or step that is positively recited herein may be explicitly excluded in the claims, whether such components, elements, attributes, or steps are listed as alternatives or whether they are recited in isolation.

The disclosures of each patent, patent application, and publication cited or described in this document are hereby incorporated herein by reference, in their entirety.

The present disclosure provides methods and systems for increasing hair growth in subjects in need thereof due to male- or female-pattern hair loss, pathological hair loss, or hair loss after injury. By any use of any appropriate mechanical, electromagnetic, or chemical means, the present disclosure pertains to the segmentation of hair follicles into two or more disunited subunits in order to form new follicles from the respective subunits. In contrast with known techniques for bisecting hair follicles and as described more fully herein, the present methods and systems permit the bisection of single follicles or populations of follicles with a high degree of efficiency, and thereby represent an effective treatment option for those in need of increased hair growth. As used herein an “increase in hair growth” refers to an increase in the number of follicles that are capable of producing hairs, preferably terminal hairs, at a body surface.

A recent study by Toscani, et al. involved the horizontal bisection and implantation of follicles and the staining of the resulting upper and lower portions of the bisected follicle for various markers known to be involved in hair follicle cycling. See Toscani M, et al., Dermatol Surg 2009; 35:1119-1125. The study revealed the possible presence in both portions of bisected follicles of a stem cell reservoir that could be capable of driving the formation of an entire hair from each portion. The instant disclosure provides methods and systems that employ a novel interpretation of data that suggests the ability of bisected follicles to generate new hair follicles from the portions of the bisected structure. The present inventors have found that bisected follicles that successfully produce new follicles is in the range of about 40% to about 70% (the upper end of the range reflecting situations whereby all bisections result two new follicles each, and whereby none of the bisections result in one or no functional follicles), and that this degree of efficiency is increased when performed in situ by the inventive methods and systems disclosed herein. Furthermore, it has been discovered that the bisection of a hair follicle (and concurrent injury of surrounding tissue) in accordance with the present disclosure may be performed in such a manner as to produce other “wound” signals that may stimulate stem cell division and differentiation from pools of epithelial stem cells, bulge stem cells, and bone marrow-derived stem cells that are present at or are recruited to the site of the hair follicle and/or surrounding tissue. Such stem cell activity may contribute to the generation of new, complete hair follicles, and result in regeneration, remodeling, resurfacing, restoration, follicular neogenesis, neocollagenesis, stem cell recruitment, activation, or differentiation, reepitheliazation, wound healing, or other desired biological or mechanical modification of the body surface and the attendant cosmetic and therapeutic benefits.

In one aspect, methods for stimulating hair growth at a body surface are provided comprising: (a) identifying a first hair follicle on the body surface; (b) segmenting the first hair follicle into at least two disunited subunits in response to the identification; (c) optionally applying a composition to the site of the first hair follicle; and (d) identifying a further hair follicle; segmenting the further hair follicle into at least two disunited subunits; and optionally applying the same or a different composition to the site of the further hair follicle, or, (e) segmenting each of one or more further hair follicles into at least two disunited subunits contemporaneously with step (b); and optionally applying the same or a different composition to the site of the further hair follicle contemporaneously with step (c).

The body surface is preferably a skin surface. Skin surfaces of all types, for example, facial skin, the scalp, or skin on the chest, legs, pubic region, or arms, may be subjected to treatment in accordance with the present disclosure.

The identification of the first follicle on the body surface may be accomplished by locating a hair that corresponds to a subsurface follicle (i.e., an indirect assessment of the location of a follicle), or by direct location of a follicle, which may be performed by a (preferably suitably trained) human being using no more than that person's eyes, or by any appropriate light- or sound-based system, such as a lens-bearing device (e.g., a microscope or other magnifier), a camera, a laser scanner, a sonar- or ultrasound-based device, a photoacoustic imager, or a fluoroscopic device. The “identification” of the first follicle may include an assessment of certain characteristics of that follicle, such as its size, depth, and angle relative to the body surface. Certain characteristics of a follicle may be ascertained by assessing a hair corresponding to that follicle. For example, a good estimate of the angle of the follicle relative to the body surface may be made based on the angle at which the hair protrudes from the body surface; hair on the forearms may protrude at a low angle relative to the surface of the arm, indicating that the underlying follicles are angled in the direction of the protruding hair, while male facial hair on the cheeks is oriented substantially perpendicular to the skin surface, corresponding to underlying follicles that are similarly oriented. As will be explained more fully below, and assessment of the angle of the follicle relative to the body surface provides information that is useful for performing the present methods.

The “identification” of the first hair follicle may comprise an assessment of general characteristics of the body surface, such as the density of hair at the portion of the body surface at which the first hair follicle is located and optionally one or more other portions of the body surface, the distribution of hair on the body surface, the orientation of at least one hair or follicle that is in addition to the first hair follicle, the absence or presence of one or more other physical features associated with the body surface, or other relevant characteristics of the body surface.

“Identification” of a first or further hair follicle may include the determination of either the absence or the presence and location of at least one physical feature. For example, an imaged portion of the body surface may be assessed to determine whether any of one or more physical features is absent or present, and if present, where that physical feature is located in the portion, where that physical feature is located relative to other known physical features (including hair follicles), or both. The portion may be selected at random or according to a predetermined pattern. The physical feature may be selected from a group of physical features that are present at the type of body surface of which the portion is a part. For example, if the body surface is the scalp, the physical feature may be a hair, a vellus hair, a hair pore, a sweat gland, an area of pigmentation, scar tissue, a wound, a “featureless” patch of skin (i.e., an area of skin without any of the preceding features), or another normal or abnormal physical feature that is known to occur at the scalp. Likewise, if the body surface is facial skin, the physical feature may be a hair, a vellus hair, a miniaturized hair, a hair pore, a sweat gland, an area of pigmentation, scar tissue, a wound, a blood vessel, a wrinkle, a wart, a “featureless” patch of skin (i.e., an area of skin without any of the preceding features), or another normal or abnormal physical feature that is known to occur on facial skin. It is to be noted that the absence of one physical feature may correspond to the presence of another physical feature. For example, the absence of a hair may correspond to the presence of a sweat gland or the presence of an area of skin without any other of the designated physical features.

Other physical features my include aging-related skin conditions, pigmentation disorders, acne, stretch marks, skin disorders (such as psoriasis, leprosy, atopic dermatitis, or other conditions resulting from an autoimmune disorder), skin infections, skin lesions, keloids.

“Aging-related skin condition” may refer to a condition resulting from intrinsic aging (i.e., chronological aging) as well as extrinsic aging (i.e., resulting from environmental conditions such as photoaging). Examples of such conditions are wrinkles (e.g., fine and coarse wrinkles), brown spots, dyspigmentation, laxity, yellow hue, telangiectasia, leathery appearance, and cutaneous malignancies. Wrinkles and skin laxity are primarily caused by a decrease in the subcutaneous fat layer combined with decreased collagen and elastin synthesis in the dermis. Alterations in skin pigmentation (e.g., brown spots and dyspigmentation) are related to altered melanocyte function and changes in melanin accumulation within basal keratinocytes. Changes in skin blood vessel dilation and distribution contribute to the appearance of telangiectasia and spider veins. Increased skin malignancies are also associated with increased skin aging and generally result from a combination of environmental exposure (i.e., high UV exposure prior to age 18) and genetics. A reduction of sweat gland number and function is another age-related skin condition.

“Pigmentation disorder” refers to a skin or hair condition arising from abnormal skin or hair pigmentation that may but need not be caused by alterations in melanocyte function or viability. Such disorders include abnormal pigmentation in humans such as albinism, melasma, vitiligo, hair graying, freckles, hemochromatosis, hemosideriosis, and tinea versicolor.

“Acne” generally refers to a skin condition arising from the pilosebaceous unit characterized by hyperkeratinization, P. acnes infection, and abnormal sebum production and that results in a visible skin lesion.

The assessment of the orientation of at least one hair or follicle that is in addition to the first hair follicle may provide an indication of a directional pattern of hair growth on the body surface. Hair whorls are patches of hair in which the individual hairs are oriented in substantially the same direction or in accordance with a collectively shared pattern. For example, clockwise or counterclockwise hair whorls occur very frequently on the heads of human males. The detection of a directional pattern of hair growth can be used to determine the direction in which treatment progresses relative to the body surface pursuant to an iterative treatment regime.

The identification of the first hair follicle may be performed using an appropriate device. The identification may include an assessment of a portion of the body surface by a human being using no more than that person's eyes. By looking at the desired portion of the body surface, a practitioner, for example, may assess one or more characteristics of the hair follicle or of the general characteristics of the body surface (as described above), or both. Alternatively or additionally, an imaging device such as a lens or camera may be used to image a desired portion of the body surface pursuant to an assessment thereof. Preferably, imaging includes the acquisition of an image of the portion and storage of the image, such as in electronic digital format. The stored image may then be used for subsequent assessments, including assessments of subparts of the image, such as the area equivalent to that which would be occupied by a hair follicle or a particular physical feature, if present. The image is preferably acquired in sufficiently high resolution to locate, distinguish among, and characterize hairs, hair follicles, other physical features, and the like. Imaging devices that are suitable for the purposes described herein may be readily identified among those skilled in the art, and may include digital cameras, charge-coupled device (CCD) cameras, or other suitable imaging systems. Other nonlimiting examples of imagers include any light- or sound-based system, such as a lens-bearing device (e.g., a microscope), a laser scanner, a sonar- or ultrasound-based device, a photoacoustic imager, or a fluoroscopic device.

In response to the identification of the first hair follicle on the body surface, the first hair follicle is segmented into at least two disunited subunits. In certain embodiments, the “identification” of a first hair follicle may comprise certain preparations for the segmentation step. The identification of the first hair follicle may comprise the positioning of the mechanism or device for segmenting the first hair follicle (for simplicity, an “incisor”, which for certain purposes may be used interchangeably with the term “incision unit”) at a suitable position for segmenting the follicle into at least two disunited subunits. The positioning of the incisor for may include horizontal and/or vertical orientation of the incisor relative to the body surface, the first hair follicle, or both. The positioning of the incisor may also or alternatively comprise orienting the incisor relative to the first hair follicle in light of any detected directional pattern of hair growth by at least one hair or follicle that is in addition to the first hair follicle.

The “identification” of the first hair follicle may comprise assessing the absence or presence of an impediment at the site of the hair follicle, and optionally displacing the impediment in response to the assessment, or selecting a location on the body surface having a preselected geometry with respect to the first hair follicle. A hair, a sweat droplet, oil, dirt, a mole, skin pigmentation, dead skin, a scab, or any combination thereof may be located at the body surface in such a manner as to constitute an impediment to assessment, segmentation, application of a composition, or any combination thereof. Even if the impediment does not interfere with assessment, segmentation, or application of a composition, it may be desirable to avoid injuring the impediment. For example, if the impediment is a hair, it may be desirable to avoid severing or otherwise damaging the hair, especially of an objective of the treatment is to promote hair growth or to increase the density of hair. This may especially be the case when treating areas of thinning hair as opposed to areas of total baldness (e.g., as in the case of female diffuse alopecia). As such, when treating to restore hair, an objective is typically not to remove hair that may already be present. Where an assessment is made that an impediment is present, it may be desirable to displace or eliminate the impediment, or to select a new location on the portion of the body surface for assessment. In other instances, it may not be necessary to address the presence of the impediment. Depending on the type of impediment that is found, any of a variety of different approaches may be used to displace or eliminate the impediment. For example, forced air may be used to blow away, blow aside, or evaporate an impediment; a hair or a sweat droplet may be blown aside, dead skin or dirt may be blown away, and a sweat droplet may be evaporated. A stream of liquid, such as water, may also be used to displace an impediment. Devices for producing forced air, a stream of liquid, or other suitable means for displacing or eliminating an impediment may be readily appreciated among those skilled in the art. Any method for displacing or eliminating an impediment may be used in accordance with the present disclosure.

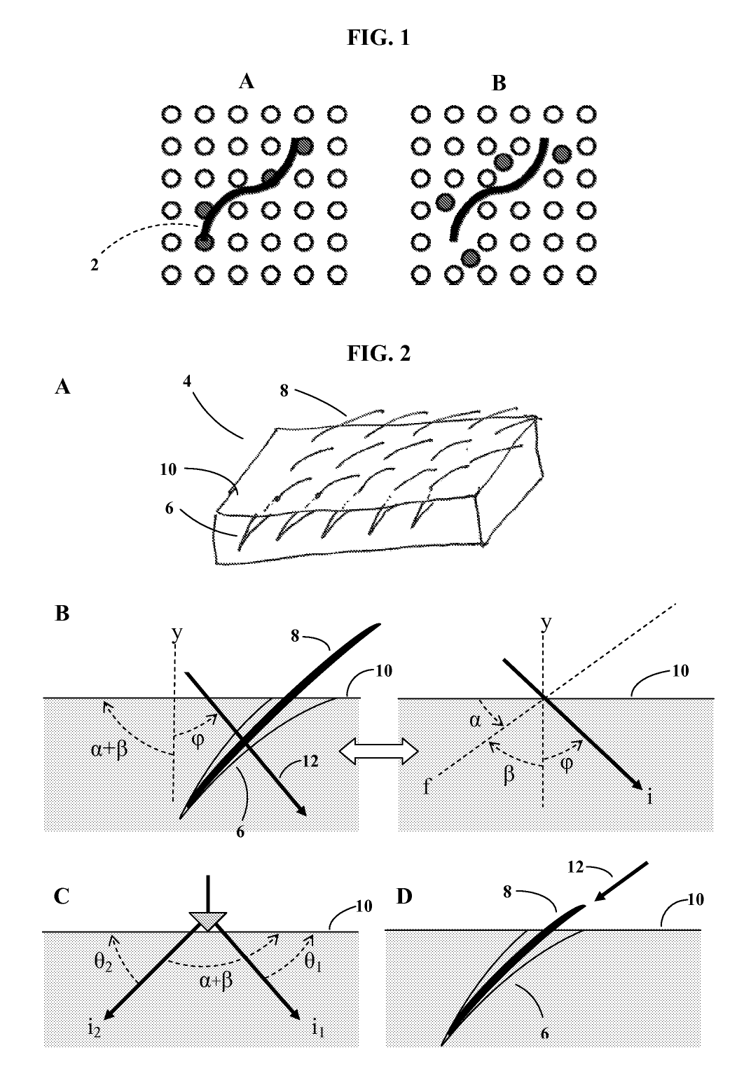

In one embodiment, a CCD camera or other digital camera can be integrated with the incision unit, e.g., a fractional laser, to automatically detect an existing hair and to redirect the trajectory of the incisor away from the hair. For example, standard imaging software such as IMAQ that runs on LabView, can readily be incorporated into an embedded micro-controller that integrates laser targeting with hair detection via the camera. In FIG. 1A, a standard fractional laser pattern is shown, wherein shaded circles (designating points on the fractional laser pattern) either clip the existing hair 2 or completely remove it. FIG. 1B shows the hair being detected and laser beam being redirected to miss the existing hair. It is also contemplated that it may be more effective and practical (from a systems integration perspective) to detect the existing hair and then selectively not fire the laser over sites that include hair. Essentially, the beam can be steered away from the hair or not fired over a hair (or any other physical feature of which injury is not desired) as appropriate.

In another embodiment (not shown), a burst of air or other gas may be used to displace an existing hair that would otherwise be compromised by the segmentation modality. A gas jet can be readily generated via a disposable CO₂ cartridge and integrated and controlled by the hair detection software in a laser integrated with a camera as described above. The embedded software via the micro-controller can gate a solenoid valve that fires the gas. An exemplary process may include (1) detection of one or more hairs; (2) firing the burst of gas to attempt to displace the hair; (3) firing the laser (or otherwise activating the incisor) selectively as described above.

If the incisor includes one or more biopsy needles or micro-needles, then gas jets could be delivered down the center lumen of the needle to displace hair distally before the needle is used to segment the hair follicle. In this example, gas-based hair displacement would not necessarily require being coupled to an imaging system; as any one needle approaches the body surface, the expelling gas will displace the hair prior to entry.

The segmentation of the first hair follicle into at least two disunited subunits is performed in response to the “identification” of the first hair follicle. Accordingly, the segmentation of the first hair follicle takes into account any assessment of the body surface, the first hair follicle, further hair follicles, impediments, or any other feature, characteristic, or condition as described above, and is in accordance with any positioning of the incisor/incision unit also as described previously.

Any suitable device or mechanism (i.e., any incisor) may be used to segment the first hair follicle into at least two disunited subunits. The segmentation may be induced by any mechanical, chemical, energetic, sound- or ultrasound-based, or electromagnetic means. Nonlimiting examples include a laser (e.g., fractional, non-fractional, ablative, or nonablative), a needle, a drilling bit, a blade, or a fluid (e.g., water or gas) jet.

The incisor may remove or ablate tissue along its trajectory (including the portion of the follicle that it intersects), or may leave such tissue substantially in place. The hair follicle may be segmented by removal of a column, slice, wedge, cube, plug, or other portion of tissue to form a “channel” that transects the follicle. The channel may extend from the body surface to a depth of about 0.5 mm to about 4 mm below the surface, or to any depth that is necessary to transect and segment the hair follicle. As described more fully herein, the channel may be oriented substantially perpendicular or at an oblique angle relative to the body surface. The removal of a column of tissue may be accomplished by any suitable technique, including a fractional ablative laser, a punch biopsy needle, a microneedle, a micro-coring needle, or another suitable modality. In other embodiments, the incisor may segment the hair follicle by any other means that does not remove a column of tissue but that otherwise segments the follicle into disunited subunits. In such instances, non-coring needles (e.g., an acupuncture needle or a sewing-type needle), blades, drilling bits or any other preferably sharp implement capable of penetrating tissue may be used, as well as non-ablative lasers, water jets, compressed air jets, and the like.

In addition to segmenting the first hair follicle, removal of a column of tissue may invoke a full thickness skin excision (FTE) model to establish a skin healing state that is conducive to follicular neogenesis by removing all tissue components and relying on de novo hair follicle formation. The channels that are formed pursuant to this type of injury are surrounded by intact skin with viable keratinocytes and melanocytes. Due to the proximity of the viable cells to the site of injury, the re-epithelialization process is more rapid than bulk ablation of tissue over a large area. The standard FTE model is created with a scalpel in animal models. This aggressive procedure does not lend itself directly to commercialization due to risk of scarring. However, various fractional laser modalities may be used to achieve this deeper disruption on a grid pattern. A fractional laser may be used to “drill”, for example, 1 mm diameter holes. Although tissue is completely removed within the 1 mm hole, the surrounding intact tissue prevents scarring and therefore the FTE model is invoked within each hole.

A fractional like hole pattern can also be achieved with punch biopsy needles. When inserted into the scalp, the cored skin samples can be removed and as in above, the FTE model is invoked within each hole. Similarly, and for smaller holes, micro needles and micro-coring needles could be used. Micro-roller needle devices already on the market, may be used to create the fractional injury. Other modalities such as ultrasound, electroporation, RF ablation, and electromagnetic fields can all be used to perturb and/or remove the tissue of a body surface such that the aforementioned models are invoked.

Electromagnetic means of follicle segmentation include, for example, use of a laser (e.g., using lasers, such as those that deliver ablative, non-ablative, fractional, non-fractional, and/or are CO₂-based, or Erbium-YAG-based, etc.). Segmentation can also be achieved through, for example, the use of visible, infrared, ultraviolet, radio, or X-ray irradiation. Electrical or magnetic means of follicle segmentation can be achieved, for example, through the application of an electrical current, or through electroporation or RF ablation. Electric or magnetic means can also include the induction of an electric or a magnetic field, or an electromagnetic field. For example, an electrical current can be induced in the skin by application of an alternating magnetic field. A radiofrequency power source can be coupled to a conducting element, and the currents that are induced will heat the follicle, resulting in follicular segmentation. Follicle segmentation can also be achieved through surgery, for example, a biopsy, a surgical incision, etc.

In some embodiments, follicle segmentation is by laser treatment. In a preferred embodiment, follicle segmentation by laser treatment is by a fractional laser, using, e.g., an Erbium-YAG laser at around 1540 nm or around 1550 nm (for example, using a Fraxel® laser (Solta Medical)). Treatment with an Erbium-YAG laser at 1540 or 1550 nm is typically non-ablative, and pinpoint bleeding typical of laser treatment is not observed since the outer portion of the body surface (for example, in skin, the stratum corneum) is left intact. The column of dead cells (for skin, epidermal and/or dermal) in the path of the laser treatment is termed a “coagulum.” In another embodiment, follicle segmentation by laser treatment is by a fractional laser, using, e.g., a CO₂ laser at 10,600 nm. Treatment with a CO₂ laser at 10,600 nm is typically ablative.

A standard CO₂ or Erbium-YAG laser can be used to accomplish follicle segmentation. Use of such lasers has an advantage making it possible to select the specific depth of body surface disruption to effectively remove the outer portions (e.g., stratum corneum) and internal portions (e.g., epidermis), or parts thereof, while segmenting the follicle.

In one embodiment, the laser treatment is ablative. For example, full ablation of tissue is generated by the targeting of tissue water at wavelengths of 10,600 nm by a CO₂ laser or 2940 nm by an Erbium-YAG laser. With respect to skin, in this mode of laser treatment the epidermis is removed entirely and the dermis receives thermal tissue damage. The depth of tissue ablation may be a full ablation of the epidermis, or a partial ablation of the epidermis, with both modes causing follicle segmentation. In another variation, the depth of ablation may extend partially into the dermis, to generate a deep wound. The denuded skin surface may then treated with a composition described infra; alternatively, the composition can be delivered into the skin after the initial re-epithelialization has occurred already, to prevent clearance and extrusion of any drug-containing depots from the tissue site by the biological debris-clearance process.

As disclosed supra, an full thickness excision model may be invoked by use of a fractional laser.

In some embodiments, the laser treatment is ablative and fractional. For example, fractional tissue ablation can be achieved using a CO₂ laser at 10,600 nm or an Erbium-YAG laser at 2940 nm (e.g., the Lux 2940 laser, Pixel laser, or Profractional laser). In some such embodiments, the lasing beam creates micro-columns of thermal injury into the body surface, at depths up to 4 mm and vaporizes the tissue and segments the follicle in the process. Ablative treatment with a fractional laser leads to ablation of a fraction of the body surface leaving intervening regions of normal tissue intact, which in skin allows for rapid repopulation of the epidermis. In one embodiment, a composition described herein is delivered into the dermis immediately after wounding, or after initial re-epithelialization has occurred.

In another embodiment, the mode of laser treatment is non-ablative, wherein outer portions of the body surface (e.g., in skin, the stratum corneum and the epidermis) are intact after treatment, with subsurface portions (e.g., dermis) selected for the deep thermal treatment contemporaneously with follicle segmentation. This can be accomplished by cooling the epidermis during the laser treatment. For example, one could use the timed cooling of the outer portions of the body surface with a cryogen spray while the laser delivers deep thermal damage to the subsurface portions. In this application, the depth of treatment may be 1 mm to 3 mm into the body surface, or any depth that is suitable for follicle segmentation. One could also use contact cooling, such as a copper or sapphire tip. Lasers that are non-ablative have emission wavelengths between 1000-1600 nm, with energy fluences that will cause thermal injury, but do not vaporize the tissue. The non-ablative lasers can be bulk, wherein a single spot beam can be used to treat a homogenous section of tissue. Lasers that are non-ablative include the pulsed dye laser (vascular), the 1064 Nd:YAG laser, or the Erbium-YAG laser at 1540 nm or 1550 nm (e.g., the Fraxel® laser). Use of an Erbium-YAG laser at around 1540 nm or around 1550 nm, as opposed to its use at 2940 nm, “coagulates” zones of dermis and epidermis (forming a “coagulum”) and leaves the stratum corneum essentially intact while segmenting the follicle.

In another embodiment, the mode of laser treatment is fractional and non-ablative. Treatment with a fractional, non-ablative laser leads to perturbation of a fraction of the body surface, segmenting the follicle while leaving intervening regions of normal tissue intact (which in skin, allows for rapid repopulation of the epidermis). Approximately 15%-25% of the body surface is treated per session. As in any non-ablative process, the barrier function is maintained, while deep thermal heating of subsurface portions can occur in order to accomplish segmentation of the follicle. For example, in skin, zones of dermis and epidermis are coagulated and the stratum corneum is left essentially intact. This process has been coined “fractional photothermolysis” and can be accomplished, e.g., using the Erbium-YAG laser with an emission at or around 1540 nm or 1550 nm. In one embodiment, a composition described herein (e.g., a lithium composition) is delivered immediately after the tissue injury, deep into the body surface (in skin, into the dermis). In another embodiment, a combination of bulk and fractional ablation modes of tissue injury are used.

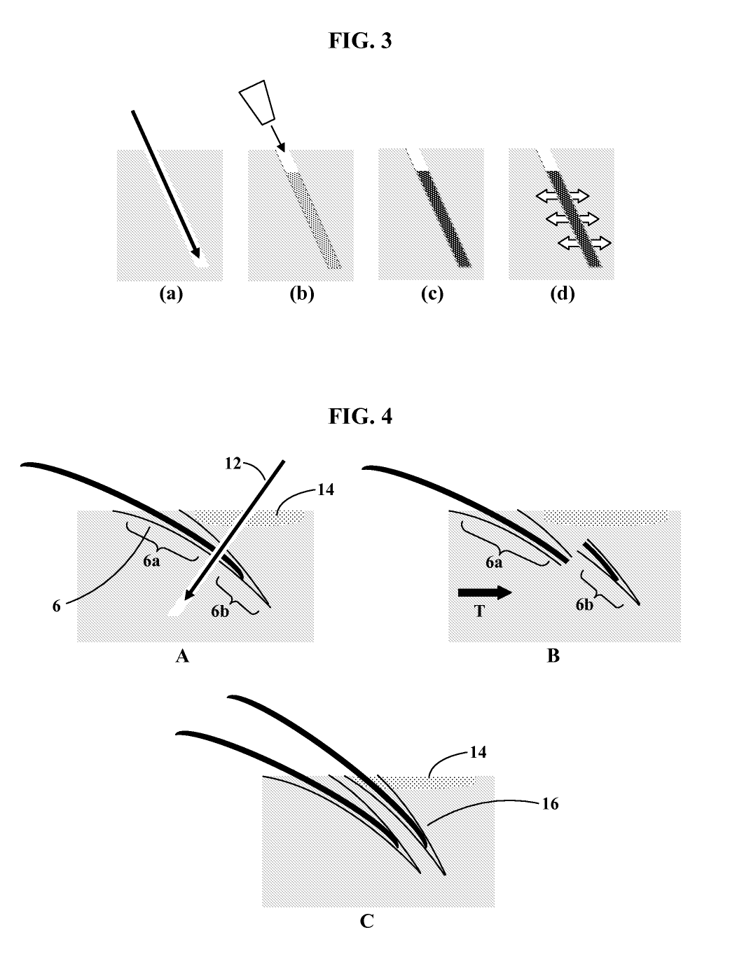

FIG. 2A depicts a portion 4 of a body surface 10 that features subsurface hair follicles 6 with hairs 8 protruding therefrom. The first hair follicle may be segmented by applying an incisor at an oblique angle relative to the body surface at the location of the follicle. The present inventors have discovered that the efficiency of a process for segmenting hair follicles is significantly improved by directing an incisor at an angle that is not “downwards”, i.e., at 90°, relative to the body surface. Certain hair follicles may have an orientation of about 90° relative to the skin, and the likelihood that such follicles will be intersected, for example, by a laser that is directed at a conventional angle relative to the skin is significantly lower than if the laser were directed at an angle in accordance with the present disclosure. As used herein, an “oblique” angle is an angle having a value relative to the most proximate body surface that is less than 90°, i.e., the oblique angle is always expressed in terms of a value that is between 0° and 89°, inclusive. In some embodiments, incisor is applied at an angle of 89°, 85°, about 80°, about 75°, about 70°, about 65°, about 60°, about 55°, about 50°, about 45°, about 40°, about 35°, about 30°, about 25°, about 20°, about 15°, about 10°, about 5°, or less relative to the body surface. Expressed differently and as depicted in FIG. 2B, the incisor 12 may be applied at an angle φ relative to axis y that is perpendicular to the body surface 10, wherein the first follicle 6 is oriented at an angle α relative to said body surface, wherein the sum of angle α and an angle β is 90°. In certain embodiments, the sum of angle φ and angle β is about 65° to about 115°. For example, the sum of angle φ and angle β may be about 70°, about 75°, about 80°, about 85°, about 90°, about 95°, about 100°, about 105°, or about 110°.

The segmentation of a first hair follicle by applying an incisor at an oblique angle relative to the body surface may alternatively comprise splicing a hair follicle substantially along its long axis. For example, given a hair follicle that is oriented at about 40° relative to the body surface, the incisor may be directed at a comparable angle against the body surface at the location of the follicle and parallel to the long axis of the follicle. This process is depicted in FIG. 2D, which shows the application of an incisor 12 to the body surface 10 at the location of a hair follicle 6 (which incidentally includes a hair 8) and at an angle that is substantially the same as the angle at which hair follicle 6 is oriented relative to the body surface 10. The application of an incisor in this manner preferably functions to splice the follicle along its long axis into at least two portions (if two portions are produced, halves). Each portion of the spliced follicle contains all of the biological follicular components that are necessary to generate a complete follicle and produce hair. Thus, the splicing of a hair follicle in this manner can generate a pair of hair-producing follicles from a single follicle.

Optionally, further to the process of segmenting at least a first hair follicle by applying an incisor at an oblique angle relative to the body surface, an incisor may also be applied substantially “downwards”, i.e., at about 90°, relative to the body surface in order to segment a further hair follicle that is oriented at a substantially similar angle relative to the body surface. The application of an incisor substantially downwards onto a hair follicle having this orientation preferably functions to splice the follicle into at least two substantially vertically oriented halves. Each half of the spliced follicle contains all of the biological follicular components that are necessary to generate a complete follicle and produce hair. Thus, the splicing of a hair follicle in this manner can generate a pair of hair-producing follicles from a single follicle.

FIG. 2C provides a guide as to how the angle of an incisor relative to a body surface 10 may be measured in accordance with the present disclosure. As will be more fully described infra, an incision unit may be configured for applying a first incisor and a second incisor to the body surface, each at an oblique angle relative to the body surface. In FIG. 2C, a first incisor i₁ is applied at an angle θ₁ relative to body surface 10. A second incisor i₂ is also applied at an angle relative to body surface 10, and although the angle at which second incisor i₂ is applied to body surface 10 could be measured as angle α+β, in accordance with the present disclosure, angle at which second incisor i₂ is applied to body surface 10 is measured as angle θ₂, i.e., relative to the most proximate body surface so that the measured angle is less than 90°.

A composition may be applied to the site of the first hair follicle. The application of a composition is optional, as it has presently been discovered that duplication of follicles may be accomplished by segmentation alone. As used herein, the “site of a hair follicle” may include the follicle itself, any tissue that is adjacent to the follicle (e.g., within about 0.5 mm, within about 1 mm, within about 1.5 mm, within about 2 mm, within about 3 mm, within about 4 mm, or within about 5 mm of the follicle), a portion of the body surface that is adjacent to the follicle (e.g., within about 0.5 mm, within about 1 mm, within about 1.5 mm, within about 2 mm, within about 3 mm, within about 4 mm, or within about 5 mm of the follicle), any channel or other site of injury that transects the hair follicle as a result of the segmentation of the follicle, or any combination thereof. The composition may be applied to the site of the first hair follicle after or contemporaneously with the segmentation of the follicle. As used herein, “contemporaneously” means that during at least part of the time that the follicle is being segmented, the composition is applied to the site of the follicle. Thus, if the segmentation occurs during a time period having a total duration of one second, applying a composition to the site of the hair follicle for 0.5 seconds after the segmentation of the follicle and for 0.1 seconds during the period of time during which segmentation takes place will be considered to have been contemporaneous with the segmentation of the hair follicle.

The composition may comprise one or more physiologically active compounds. For example, the composition may include one or more of compounds that can influence the generation of hair follicles or the stimulation of hair growth, antioxidants, antihistamines, anti-inflammatory agents, anti-cancer agents, retinoids, anti-androgen agents, immunosuppressants, channel openers, antimicrobials, herbs, extracts, vitamins, co-factors, psoralen, anthralin, and antibiotics. The type of composition that is applied to the site of the follicle, the manner of application, or both may be selected from a set of compositions and methods of application that are appropriate for use with the type of injury to which the site of the follicle was subjected.

Any compound or composition that can release a lithium ion is suitable for use in the present methods and systems. Such compounds include but are not limited to a pharmaceutically acceptable prodrug, salt or solvate (e.g., a hydrate) of lithium (sometimes referred to herein as “lithium compounds”). Optionally, the lithium compounds can be formulated with a pharmaceutically acceptable vehicle, carrier, diluent, or excipient, or a mixture thereof. Additionally, lithium-polymer complexes can be utilized to developed various sustained release lithium matrices.

Any form of lithium approved for pharmacological use may be used. For example, lithium is best known as a mood stabilizing drug, primarily in the treatment of bipolar disorder, for which lithium carbonate (Li₂CO₃), sold under several trade names, is the most commonly used. Other commonly used lithium salts include lithium citrate (Li₃C₆H₅O₇), lithium sulfate (Li₂SO₄), lithium aspartate, and lithium orotate. A lithium formulation well-suited for use in the composition is lithium gluconate, for example, a topical ointment of 8% lithium gluconate (Lithioderm™), is approved for the treatment of seborrhoeic dermatitis. See, e.g., Dreno and Moyse, 2002, Eur J Dermatol 12:549-552; Dréno et al., 2007, Ann Dermatol Venereol 134:347-351 (abstract); and Ballanger et al., 2008, Arch Dermatol Res 300:215-223, each of which is incorporated by reference herein in its entirety. Another lithium formulation is lithium succinate, for example, an ointment comprising 8% lithium succinate, which is also used to treat seborrhoeic dermatitis. See, e.g., Langtry et al., 1996, Clinical and Experimental Dermatology 22:216-219; and Cuelenaere et al., 1992, Dermatology 184:194-197, each of which is incorporated by reference herein in its entirety. In one embodiment, the lithium formulation is an ointment comprising 8% lithium succinate and 0.05% zinc sulfate (marketed in the U.K. as Efalith). See, e.g., Efalith Multicenter Trial Group, 1992, J Am Acad Dermatol 26:452-457, which is incorporated by reference herein in its entirety. Examples of lithium succinate formulations and other lithium formulations for use in the intermittent lithium treatments or pulse lithium treatment described herein are also described in U.S. Pat. No. 5,594,031, issued Jan. 14, 1997, which is incorporated herein by reference in its entirety.

Any pharmaceutically acceptable lithium salt may be used. It will be understood by one of ordinary skill in the art that pharmaceutically acceptable lithium salts are preferred. See, e.g., Berge et al., J. Pharm. Sci. 1977, 66:1-19; Stahl & Wermuth, eds., 2002, Handbook of Pharmaceutical Salts, Properties, and Use, Zurich, Switzerland: Wiley-VCH and VHCA; Remington's Pharmaceutical Sciences, 1990, 18^{th} eds., Easton, Pa.: Mack Publishing; Remington: The Science and Practice of Pharmacy, 1995, 19^{th} eds., Easton, Pa.: Mack Publishing.

In some embodiments, the compositions comprise mixtures of one or more lithium salts. For example, a mixture of a fast-dissolving lithium salt can be mixed with a slow dissolving lithium salt proportionately to achieve the release profile. In certain embodiments, the lithium salts do not comprise lithium chloride.

In some embodiments, the lithium salt can be the salt form of anionic amino acids or poly(amino) acids. Examples of these are glutamic acid, aspartic acid, polyglutamic acid, polyaspartic acid.

By reciting lithium salts of the acids set forth above, it is not intended to mean only the lithium salts prepared directly from the specifically recited acids. In contrast, the present disclosure encompasses the lithium salts of the acids made by any method known to one of ordinary skill in the art, including but not limited to acid-base chemistry and cation-exchange chemistry.

In another embodiment, lithium salts of anionic drugs that positively affect hair growth, such as prostaglandins can be administered. In another embodiment, a large anion or multianionic polymer such as polyacrylic acid can be complexed with lithium, then complexed with a cationic compound, such as finasteride, to achieve a slow release formulation of both lithium ion and finasteride. Similarly, a lithium complex with a polyanion can be complexed further with the amines of minoxidil, at pHs greater than 5.

Lithium compounds for use herein may contain an acidic or basic moiety, which may also be provided as a pharmaceutically acceptable salt. See, Berge et al., J. Pharm. Sci. 1977, 66:1-19; Stahl & Wermuth, eds., 2002, Handbook of Pharmaceutical Salts, Properties, and Use Zurich, Switzerland: Wiley-VCH and VHCA.

In some embodiments, the lithium salts are organic lithium salts. Organic lithium salts for use in these embodiments include lithium 2,2-dichloroacetate, lithium salts of acylated amino acids (e.g., lithium N-acetylcysteinate or lithium N-stearoylcysteinate), a lithium salt of poly(lactic acid), a lithium salt of a polysaccharides or derivative thereof, lithium acetylsalicylate, lithium adipate, lithium hyaluronate and derivatives thereof, lithium polyacrylate and derivatives thereof, lithium chondroitin sulfate and derivatives thereof, lithium stearate, linoleic acid, lithium lenoleate, lithium oleate, lithium taurocholate, lithium cholate, lithium glycocholate, lithium deoxycholate, lithium alginate and derivatives thereof, lithium ascorbate, lithium L-aspartate, lithium benzenesulfonate, lithium benzoate, lithium 4-acetamidobenzoate, lithium (+)-camphorate, lithium camphorsulfonate, lithium (+)-(1S)-camphor-10-sulfonate, lithium caprate, lithium caproate, lithium caprylate, lithium cinnamate, lithium citrate, lithium cyclamate, lithium cyclohexanesulfamate, lithium dodecyl sulfate, lithium ethane-1,2-disulfonate, lithium ethanesulfonate, lithium 2-hydroxy-ethanesulfonate, lithium formate, lithium fumarate, lithium galactarate, lithium gentisate, lithium glucoheptonate, lithium D-gluconate, lithium D-glucuronate, lithium L-glutamate, lithium α-oxoglutarate, lithium glycolate, lithium hippurate, lithium (+)-L-lactate, lithium (±)-DL-lactate, lithium lactobionate, lithium laurate, lithium (−)-L-malate, lithium maleate, lithium malonate, lithium (±)-DL-mandelate, lithium methanesulfonate, lithium naphthalene-2-sulfonate, lithium naphthalene-1,5-disulfonate, lithium 1-hydroxy-2-naphthoate, lithium nicotinate, lithium oleate, lithium orotate, lithium oxalate, lithium palmitate, lithium pamoate, lithium L-pyroglutamate, lithium saccharate, lithium salicylate, lithium 4-amino-salicylate, sebacic acid, lithium stearate, lithium succinate, lithium tannate, lithium (+)-L-tartarate, lithium thiocyanate, lithium p-toluenesulfonate, lithium undecylenate, or lithium valerate. In some embodiments, the organic lithium salt for use in these embodiments is lithium (S)-2-alkylthio-2-phenylacetate or lithium (R)-2-alkylthio-2-phenylacetate (e.g., wherein the alkyl is C2-C22 straight chain alkyl, preferably C8-16). See, e.g., International Patent Application Publication No. WO 2009/019385, published Feb. 12, 2009, which is incorporated herein by reference in its entirety.

The organic lithium salts may comprise the lithium salts of acetic acid, 2,2-dichloroacetic acid, acetylsalicylic acid, acylated amino acids, adipic acid, hyaluronic acid and derivatives thereof, polyacrylic acid and derivatives thereof, chondroitin sulfate and derivatives thereof, poly(lactic acid-co-glycolic acid), poly(lactic acid), poly(glycolic acid), pegylated lactic acid, stearic acid, linoleic acid, oleic acid, taurocholic acid, cholic acid, glycocholic acid, deoxycholic acid, alginic acid and derivatives thereof, anionic derivatives of polysaccharides, poly(sebacic anhydride)s and derivatives thereof, ascorbic acid, L-aspartic acid, benzenesulfonic acid, benzoic acid, 4-acetamidobenzoic acid, (+)-camphoric acid, camphorsulfonic acid, (+)-(1S)-camphor-10-sulfonic acid, capric acid, caproic acid, caprylic acid, cinnamic acid, citric acid, cyclamic acid, cyclohexanesulfamic acid, dodecylsulfuric acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucuronic acid, L-glutamic acid, α-oxoglutaric acid, glycolic acid, hippuric acid, (+)-L-lactic acid, (±)-DL-lactic acid, lactobionic acid, lauric acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinic acid, oleic acid, orotic acid, oxalic acid, palmitic acid, pamoic acid, L-pyroglutamic acid, saccharic acid, salicylic acid, 4-amino-salicylic acid, sebacic acid, stearic acid, succinic acid, tannic acid, (+)-L-tartaric acid, thiocyanic acid, p-toluenesulfonic acid, undecylenic acid, or valeric acid. Other organic lithium salts for use in these embodiments is the lithium salt of (S)-2-alkylthio-2-phenylacetic acid or the lithium salt of (R)-2-alkylthio-2-phenylacetic acid (e.g., wherein the alkyl is C2-C22 straight chain alkyl, preferably C8-16). See, e.g., International Patent Application Publication No. WO 2009/019385, published Feb. 12, 2009, which is incorporated herein by reference in its entirety.

In some embodiments of the present compositions, the organic lithium salt can be modified to create sustained release lithium salts. Due to the size of the lithium ion, it is possible that the residence time of ion at the treatment site will be short. In efforts to generate sustained release lithium salts, the hydrophobicity of the salt can be enhanced and made “lipid-like,” to, for example, lower the rate of ionization of the salt into lithium ions. For example, lithium chloride has a much faster rate of ionizing into lithium ions, than lithium stearate or lithium orotate. In that regard, the lithium salt can be that of a cholesterol derivative, or a long chain fatty acids or alcohols. Lipid complexed lithium salts of size less than 10 microns can also be effectively targeted to the hair follicles and “tethered” to the sebaceous glands, by hydrophobic-hydrophobic interactions.

In some embodiments, the organic lithium salt can be in the form of complexes with anionic compounds or anionic poly(amino acids) and other polymers. The complexes can be neutral, wherein all of the negative charges of the complexation agent are balanced by equimolar concentrations of Li ions. The complexes can be negatively charged, with lithium ions bound to an anionic polymer. The complexes can be in the form of nano-complexes, or micro-complexes, small enough to be targeted to the hair follicles. If the complexes are targeted to the dermis, the charged nature of the complexes will “tether” the complexes to the positively charged collagen. This mode of tethering holds the Li ions at the site of delivery, thereby hindering fast in-vivo clearance. Examples of negatively charged polymers that may be used are poly(acrylates) and its copolymers and derivatives thereof, hyaluronic acid and its derivatives, alginate and its derivatives, etc. In one variation, the anionic lithium complexes formed as described above can be further complexed with a cationic polymer such as chitosan, or polyethylimine form cell-permeable delivery systems.

The lithium salt can be that of a fatty acid, e.g., lithium stearate, thereby promoting absorption through skin tissues and extraction into the lipid compartments of the skin. In another example, the lithium salt of sebacic acid can be administered to the skin for higher absorption and targeting into structures of the skin, such as hair follicles.

The lithium salts may be inorganic lithium salts. Inorganic lithium salts for use in these embodiments include halide salts, such as lithium bromide, lithium chloride, lithium fluoride, or lithium iodide. In one embodiment, the inorganic lithium salt is lithium fluoride. In another embodiment, the inorganic lithium salt is lithium iodide. In certain embodiments, the lithium salts do not comprise lithium chloride. Other inorganic lithium salts for use in these embodiments include lithium borate, lithium nitrate, lithium perchlorate, lithium phosphate, or lithium sulfate.

The inorganic lithium salts may comprise the lithium salts of boric acid, hydrobromic acid, hydrochloric acid, hydrofluoric acid, hydroiodic acid, nitric acid, perchloric acid, phosphoric acid, or sulfuric acid.

Compositions containing one or more lithium compounds may be formulated with a pharmaceutically acceptable carrier (also referred to as a pharmaceutically acceptable excipients), i.e., a pharmaceutically-acceptable material, composition, or vehicle, such as a liquid or solid filler, diluent, solvent, an encapsulating material, or a complexation agent. In one embodiment, each component is “pharmaceutically acceptable” in the sense of being chemically compatible with the other ingredients of a pharmaceutical formulation, and biocompatible, when in contact with the biological tissues or organs of humans and animals without excessive toxicity, irritation, allergic response, immunogenicity, or other problems or complications, commensurate with a reasonable benefit/risk ratio. See, Remington: The Science and Practice of Pharmacy, 2005, 21st ed., Philadelphia, Pa.: Lippincott Williams & Wilkins; Rowe et al., eds., 2005, Handbook of Pharmaceutical Excipients, 5th ed., The Pharmaceutical Press and the American Pharmaceutical Association; Ash & Ash eds., 2007, Handbook of Pharmaceutical Additives, 3rd ed., Gower Publishing Company; Gibson ed., 2009, Pharmaceutical Preformulation and Formulation, 2nd ed., Boca Raton, Fla.: CRC Press LLC, each of which is incorporated herein by reference.

Suitable excipients are well known to those skilled in the art, and non-limiting examples of suitable excipients are provided herein. Whether a particular excipient is suitable for incorporation into a composition depends on a variety of factors well known in the art, including, but not limited to, the method of administration. For example, forms for topical administration such as a cream may contain excipients not suited for use in transdermal or intravenous administration. The suitability of a particular excipient depends on the specific active ingredients in the dosage form. Exemplary, non-limiting, pharmaceutically acceptable carriers for use in the lithium formulations described herein are the cosmetically acceptable vehicles provided in International Patent Application Publication No. WO 2005/120451, which is incorporated herein by reference in its entirety.

Lithium-containing compositions may be formulated to include an appropriate aqueous vehicle, including, but not limited to, water, saline, physiological saline or buffered saline (e.g., phosphate buffered saline (PBS)), sodium chloride for injection, Ringers for injection, isotonic dextrose for injection, sterile water for injection, dextrose lactated Ringers for injection, sodium bicarbonate, or albumin for injection. Suitable non-aqueous vehicles include, but are not limited to, fixed oils of vegetable origin, castor oil, corn oil, cottonseed oil, olive oil, peanut oil, peppermint oil, safflower oil, sesame oil, soybean oil, hydrogenated vegetable oils, hydrogenated soybean oil, and medium-chain triglycerides of coconut oil, lanolin oil, lanolin alcohol, linoleic acid, linolenic acid and palm seed oil. Suitable water-miscible vehicles include, but are not limited to, ethanol, wool alcohol, 1,3-butanediol, liquid polyethylene glycol (e.g., polyethylene glycol 300 and polyethylene glycol 400), propylene glycol, glycerin, N-methyl-2-pyrrolidone (NMP), N,N-dimethylacetamide (DMA), and dimethyl sulfoxide (DMSO).

Lithium-containing compositions for use in the methods and systems disclosed herein may also be formulated with one or more of the following additional agents. Suitable antimicrobial agents or preservatives include, but are not limited to, alkyl esters of p-hydroxybenzoic acid, hydantoins derivatives, propionate salts, phenols, cresols, mercurials, phenyoxyethanol, benzyl alcohol, chlorobutanol, methyl and propyl p-hydroxybenzoates, thimerosal, benzalkonium chloride (e.g., benzethonium chloride), butyl, methyl- and propyl-parabens, sorbic acid, and any of a variety of quarternary ammonium compounds. Suitable isotonic agents include, but are not limited to, sodium chloride, glycerin, and dextrose. Suitable buffering agents include, but are not limited to, phosphate, glutamate and citrate. Suitable antioxidants are those as described herein, including ascorbate, bisulfite and sodium metabisulfite. Suitable local anesthetics include, but are not limited to, procaine hydrochloride, lidocaine and salts thereof, benzocaine and salts thereof and novacaine and salts thereof. Suitable suspending and dispersing agents include but are not limited to sodium carboxymethylcelluose (CMC), hydroxypropyl methylcellulose (HPMC), polyvinyl alcohol (PVA), and polyvinylpyrrolidone (PVP). Suitable emulsifying agents include but are not limited to, including polyoxyethylene sorbitan monolaurate, polyoxyethylene sorbitan monooleate 80, and triethanolamine oleate. Suitable sequestering or chelating agents include, but are not limited to, EDTA. Suitable pH adjusting agents include, but are not limited to, sodium hydroxide, hydrochloric acid, citric acid, and lactic acid. Suitable complexing agents include, but are not limited to, cyclodextrins, including α-cyclodextrin, β-cyclodextrin, hydroxypropyl-β-cyclodextrin, sulfobutylether-β-cyclodextrin, and sulfobutylether 7-β-cyclodextrin (CAPTISOL®, CyDex, Lenexa, Kans.).

Soothing preparations, e.g., for topical administration, may contain sodium bicarbonate (baking soda), and coal tar based products. Formulations may also optionally contain a sunscreen or other skin protectant, or a waterproofing agent.

A product for application to the scalp or face may additionally be formulated so that it has easy rinsing, minimal skin/eye irritation, no damage to existing hair, has a thick and/or creamy feel, pleasant fragrance, low toxicity, good biodegradability, and a slightly acidic pH (pH less than 7), since a basic environment weakens the hair by breaking the disulfide bonds in hair keratin.

In particular embodiments, commercially available preparations of lithium can be used, such as, e.g., lithium gluconate, 8% lithium gluconate (Lithioderm™), approved for the treatment of seborrhoeic dermatitis (see, e.g., Dreno and Moyse, 2002, Eur J Dermatol 12:549-552; Dréno et al., 2007, Ann Dermatol Venereol 134:347-351 (abstract); and Ballanger et al., 2008, Arch Dermatol Res 300:215-223, each of which is incorporated by reference herein in its entirety); 8% lithium succinate (see, e.g., Langtry et al., 1996, Clinical and Experimental Dermatology 22:216-219; and Cuelenaere et al., 1992, Dermatology 184:194-197, each of which is incorporated by reference herein in its entirety); or 8% lithium succinate with 0.05% zinc sulfate (marketed in the U.K. as Efalith; see, e.g., Efalith Multicenter Trial Group, 1992, J Am Acad Dermatol 26:452-457, which is incorporated by reference herein in its entirety).

Certain lithium compounds are known to function as modulators of GSK3β (glycogen synthase kinase-3 beta). Other GSK3β modulators may be used as a physiologically active compound in accordance with the present compositions. Nonlimiting examples include: antibodies to GSK3β; 6-bromo-indirubin-3′-oxime (6-BIO); CHIR99021 (developed by Chiron, Emeryville, Calif.) (i.e., 6-[(2-{[4-(2,4-dichlorophenyl) 5-(4-methylimidazol-2-yl)pyrimidin-2-yl]am-ino}ethyl)amino]pyridine-3-carbonitrile); ARA014418 (AstraZeneca) (i.e., 4-(4-methoxybenzyl)-n′-(5-nitro-1,3-thiazol-2-yl)urea); TDZD-8 Noscira (Neuropharma) (i.e., 4-benzyl-2-methyl-1,2,4-thiadiazolidine-3,5-dione); “Compound 12” (i.e., 2-thio(3-iodobenzyl)-5-(1-pyridyl)-[1,3,4]-oxadiazole); and any combination thereof.

Still other GSK3β modulators may be used as a physiologically active compound in accordance with the present compositions. Further exemplary GSK3β modulators are listed below in Table 1.

TABLE 1

| Class or | | |
|---|---|---|
| Compound Name | Exemplary Compounds (if applicable) | Comments |
| Indirubin derivatives | 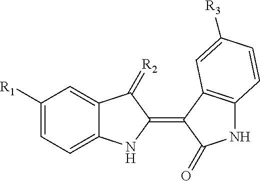 | 5- chloroindirubin (7) and indirubin 3 ′-monoxime (8) have better pharmacological properties and reduced toxicity |
| | Indirubines | |
| | 6 R₁ = R₃ = H R₂ = O | |
| | 7 R₁ = H R₂ = O R₃ = Cl | |
| | 8 R₁ = R₃ = H R₂ = NOH | |
| | 9 R₁ = R₃ = Br R₂ = O | |
| | 10 R₁= H R₂ = NOH R₃ = SO₃Na | |
| | 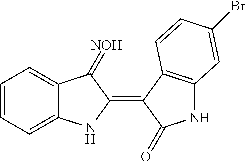 | |
| | BIO | |
| | 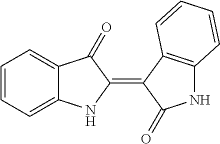 | |
| | 1 (Indirubin) | |
| | 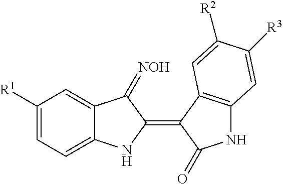 | |
| Kenpaullone and alsterpaullone | 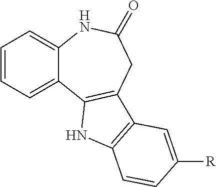 | |
| | 4 Kenpaullone R = Br | |
| | 5 Alsterpaullone R = NO₂ | |
| Purine Derivatives | 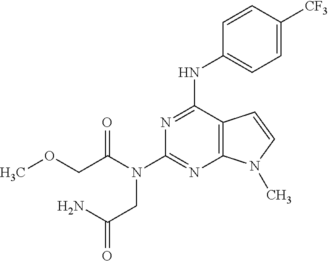 | Other Chiron compounds: CHIR 118637; CHIR 9803; CHIR 99021; CT 98023; CY 20026 |
| | 2 | |
| CHIR 9803 | 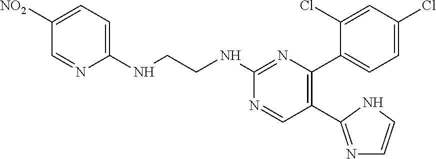 | |
| aminopyridine derivative | 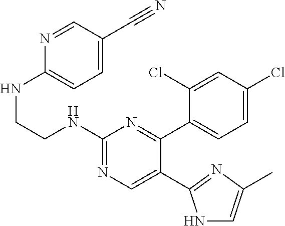 | |
| | CHIR99021 | |
| Core IS Maleimides- Bisindolylmaleimide derivatives of staurosporine | 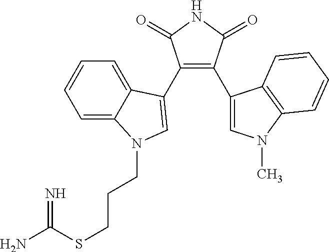 | |
| | 12 Ro 31-8220 | |
| | 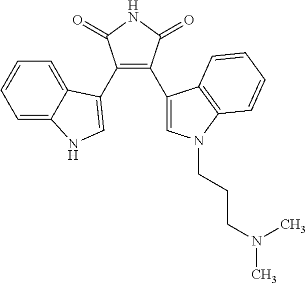 | |
| | 11 GF 109203X | |
| Core IS Maleimides | 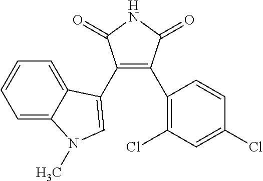 | |
| | SB-216763 19 | |
| | 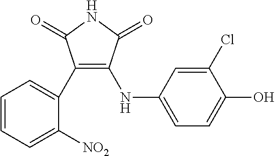 | |
| | SB-415286 20 | |
| AR A014418 | 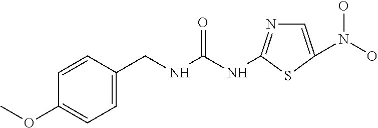 | |
| NNC 570558 | 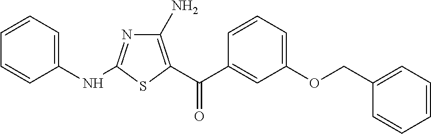 | |
| XD 4241 | Structure not known | Compound is |
| | | available for |
| | | licensing from |
| | | Xcellsyz, Ltd. |

The physiologically active compound for use in the present compositions can be a BMP inhibitor, such as the LDN-193189 small molecule (developed by Massachusetts General Hospital/Harvard); Dorsomorphin (pictured below)

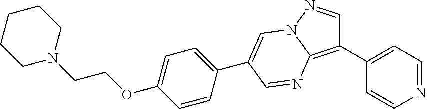

### or Dorsomorphin HCl; or, Noggin Protein (Stemgent, Cambridge, Mass.).

Other physiologically active compounds that may be used in the present compositions include Wnt modulators. For example, klotho is a protein that has been found to bind and inhibit Wnt interactions with Wnt-Receptor. See, e.g., Liu, H, et al., Science, Vol. 317. no. 5839, pp. 803-806, 10 Aug. 2007. Known Wnt agonists include 2-amino-4-(3,4-(methylenedioxy)benzylamino)-6-(3-methoxyphenyl)pyrimidine (see Osteoarthritis Cartilage. 2004 June; 12(6): 497-505) and a “group of thiophene-pyrimidines” that were identified in an academic screen for drugs that induce pancreatic beta-cell expansion (see Proc Natl Acad Sci USA. 2009 February 3; 106(5): 1427-32). These and any other Wnt modulators may be used in the present compositions.

Stem-cell signaling drug molecules may be encapsulated in matrices that are highly hydrophilic and charged, preferably linked to the dermis by covalent or ionic bonding to prevent the matrices from being cleared by phagocytosis, as part of the wound healing process.

The physiologically active compound can be a small molecule EGFR inhibitor, or metabolite thereof (e.g., a non-naturally occurring nitrogen-containing heterocycle of less than about 2,000 daltons, leflunomide, gefitinib, erlotinib, lapatinib, canertinib, vandetanib, CL-387785, PKI166, pelitinib, HKI-272, and HKI-357), EGF, an EGFR antibody (zalutumumab, cetuximab, IMC 11F8, matuzumab, SC 100, ALT 110, PX 1032, BMS599626, MDX 214, and PX 1041), a suppressor of the expression of a Wnt protein in the hair follicle or an inducer of expression of a Dkk1 protein (e.g., from lithium chloride, a molecule that synergizes with lithium chloride, the agonists 6-bromoindirubin-3′-oxime, deoxycholic acid, a pyrimidine derivative, antagonists quercetin, ICG-001, the purine derivative QS11, fungal derivatives PKF115-854 and CGPO49090, and the organic molecule NSC668036), a modulator the retinoic acid signaling pathway (trans-retinoic acid, N-retinoyl-D-glucosamine, and seletinoid G), a modulator of the estrogen signaling pathway (e.g., 17β-estradiol and selective estrogen receptor modulators), a compound which modulates the ubiquitin-proteasome system, a compound which modulates cytokine signaling of Imiquimod or IL-1alpha, a modulator of melanocortin signaling, tyrosinase activity, apoptosis signaling, endothelin signaling, nuclear receptor signaling, TGFβ-SMAD signaling, bone morphogenetic protein signaling, stem cell factor signaling, androgen signaling, retinoic acid signaling, peroxisome proliferator-activated response receptor signaling, estrogen signaling, cytokine signaling, growth factor signaling, nonandrogenic hormone signaling, toll-like receptor signaling, and neurotrophin, neuroendocine signaling, and cytokine signaling, benzoyl peroxide, a photosenitizer (e.g., aminolevulinic acid), an interferon, dacarbazine, interleukin-2, imiquimod, or a promoter of the expression of the transcription factor MITF.

The phrase “small molecule EGFR inhibitor” refers to a molecule that inhibits the function of one or more EGFR family tyrosine kinases. Tyrosine kinases of the EGFR family include EGFR, HER-2, and HER-4 (see Raymond et al., Drugs 60(Suppl.1):15 (2000); and Harari et al., Oncogene 19:6102 (2000)). Small molecule EGFR inhibitors include, for example, gefitinib (Baselga et al., Drugs 60(Suppl. 1):33 (2000)), erlotinib (Pollack et al., J. Pharm. Exp. Ther. 291:739 (1999)), lapatinib (Lackey et al., 92^{nd} AACR Meeting, New Orleans, abstract 4582 (2001)), canertinib (Bridges et al., Curr. Med. Chem. 6:825 (1999)), vandetanib (Wedge et al., Cancer Res. 62:4645 (2002)), CL-387785 (Discafani et al., Biochem. Pharmacol. 57:917 (1999)), PKI166 (Takada et al., Drug Metab. Dispos. 32:1272 (2004)), pelitinib (Torrance et al., Nature Medicine 6:1024 (2000)), HKI-272, HKI-357 (for HKI-272 and HKI-357 see, for example, Greenberger et al., 11^{th} NCI-EORTC-AACR Symposium on New Drugs in Cancer Therapy, Amsterdam, abstract 388 (2000); Rabindran et al., Cancer Res. 64:3958 (2004); Holbro et al., Ann. Rev. Pharm. Tox. 44:195 (2004); Tsou et al., J. Med. Chem. 48:1107 (2005); and Tejpar et al., J. Clin. Oncol. ASCO Annual Meeting Proc. 22:3579 (2004)), and leflunomide (Kochhar et al., FEBS Lett. 334:161 (1993)). The structures for each of these compounds is provided below in Table 2.

TABLE 2
EGFR Inhibitors

| Drug | Structure |
|---|---|
| leflunomide | 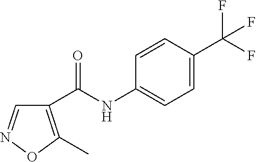 |
| Gefitinib | 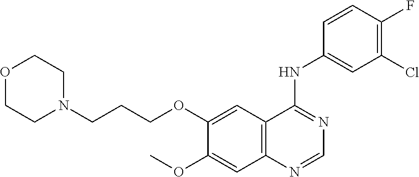 |
| Erlotinib | 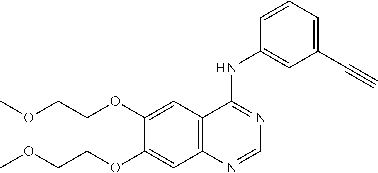 |
| Lapatinib | 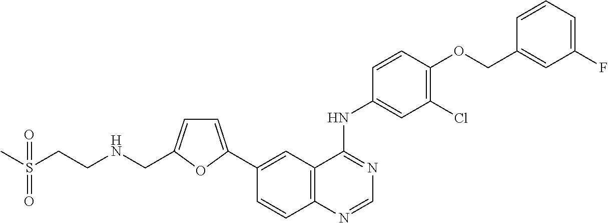 |
| Canertinib | 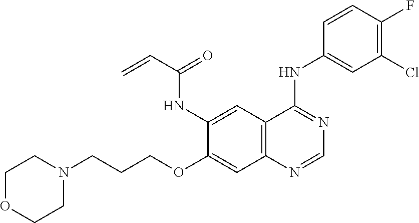 |
| Vandetanib | 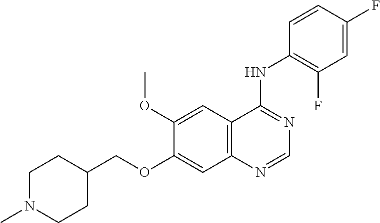 |
| CL-387785 | 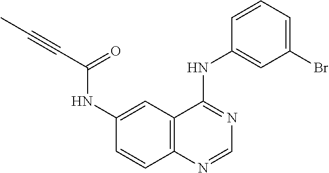 |
| PKI166 | 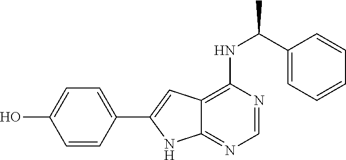 |
| Pelitinib | 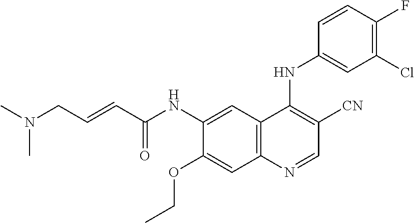 |
| HKI-272 | 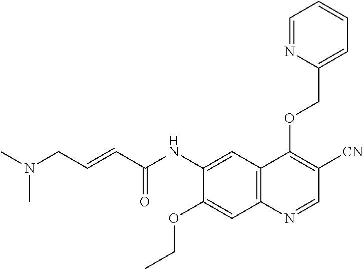 |
| HKI-357 | 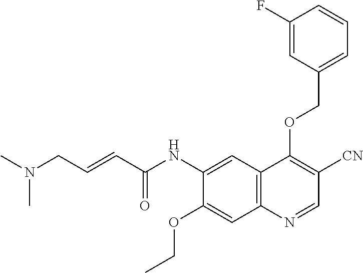 |

Small molecule EGFR inhibitors that can be used in the present compositions include anilinoquinazolines, such as gefitinib, erlotinib, lapatinib, canertinib, vandetanib, and CL-387785 and the other anilinoquinazolines disclosed in PCT Publication No. WO/2005/018677 and U.S. Pat. Nos. 5,747,498 and 5,457,105; quinoline-3-carbonitriles, such as pelitinib, HKI-272, and HKI-357, and the quinoline-3-carbonitriles disclosed in U.S. Pat. Nos. 6,288,082 and 6,002,008; pyrrolopyrimidines, such as PKI166, and the pyrrolopyrimidines disclosed in U.S. Pat. No. 6,713,474 and U.S. Patent Publication Nos. 20060211678, 20060035912, 20050239806, 20050187389, 20050165029, 20050153989, 20050037999, 20030187001, and 20010027197; pyridopyrimidines, such as those disclosed in U.S. Pat. Nos. 5,654,307 and 6,713,484; pyrazolopyrimidines, such as those disclosed in U.S. Pat. Nos. 6,921,763 and 6,660,744 and U.S. Patent Publication Nos. 20060167020, 20060094706, 20050267133, 20050119282, 20040006083, and 20020156081; isoxazoles, such as leflunomide; imidazoloquinazolines, pyrroloquinazolines, and pyrazoloquinazolines. Preferably, the small molecule EGFR inhibitor contains a heterobicyclic or heterotricyclic ring system. Each of the patent publications listed above is incorporated herein by reference.

A77 7628 refers to the active metabolite of leflunomide having the structure below.

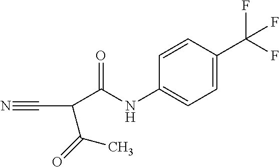

Useful antioxidants may include, without limitation, thiols (e.g., aurothioglucose, dihydrolipoic acid, propylthiouracil, thioredoxin, glutathione, cysteine, cystine, cystamine, thiodipropionic acid), sulphoximines (e.g., buthionine-sulphoximines, homo-cysteine-sulphoximine, buthionine-sulphones, and penta-, hexa- and heptathionine-sulphoximine), metal chelators (e.g, α-hydroxy-fatty acids, palmitic acid, phytic acid, lactoferrin, citric acid, lactic acid, and malic acid, humic acid, bile acid, bile extracts, bilirubin, biliverdin, EDTA, EGTA, and DTPA), vitamins (e.g., vitamin E, vitamin C, ascorbyl palmitate, Mg ascorbyl phosphate, and ascorbyl acetate), phenols (e.g., butylhydroxytoluene, butylhydroxyanisole, ubiquinol, nordihydroguaiaretic acid, trihydroxybutyrophenone), benzoates (e.g., coniferyl benzoate), uric acid, mannose, propyl gallate, selenium (e.g., selenium-methionine), stilbenes (e.g., stilbene oxide and trans-stilbene oxide), and combinations thereof.

Antioxidants that may be incorporated into the formulations of the invention include natural antioxidants prepared from plant extracts, such as extracts from aloe vera; avocado; chamomile; echinacea; ginko biloba; ginseng; green tea; heather; jojoba; lavender; lemon grass; licorice; mallow; oats; peppermint; St. John's wort; willow; wintergreen; wheat wild yam extract; marine extracts; and mixtures thereof.

The total amount of antioxidant included in the formulations can be from 0.001% to 3% by weight, preferably 0.01% to 1% by weight, in particular 0.05% to 0.5% by weight, based on the total weight of the formulation.

The composition that is applied to the site of the hair follicle may include one or more antihistamines. Exemplary antihistamines include, without limitation, Ethanolamines (e.g., bromodiphenhydramine, carbinoxamine, clemastine, dimenhydrinate, diphenhydramine, diphenylpyraline, and doxylamine); Ethylenediamines (e.g., pheniramine, pyrilamine, tripelennamine, and triprolidine); Phenothiazines (e.g., diethazine, ethopropazine, methdilazine, promethazine, thiethylperazine, and trimeprazine); Alkylamines (e.g., acrivastine, brompheniramine, chlorpheniramine, desbrompheniramine, dexchlorpheniramine, pyrrobutamine, and triprolidine); piperazines (e.g., buclizine, cetirizine, chlorcyclizine, cyclizine, meclizine, hydroxyzine); Piperidines (e.g., astemizole, azatadine, cyproheptadine, desloratadine, fexofenadine, loratadine, ketotifen, olopatadine, phenindamine, and terfenadine); and Atypical antihistamines (e.g., azelastine, levocabastine, methapyrilene, and phenyltoxamine). Both non-sedating and sedating antihistamines may be employed. Non-sedating antihistamines include loratadine and desloratadine. Sedating antihistamines include azatadine, bromodiphenhydramine; chlorpheniramine; clemizole; cyproheptadine; dimenhydrinate; diphenhydramine; doxylamine; meclizine; promethazine; pyrilamine; thiethylperazine; and tripelennamine.

Other suitable antihistamines include acrivastine; ahistan; antazoline; astemizole; azelastine; bamipine; bepotastine; bietanautine; brompheniramine; carbinoxamine; cetirizine; cetoxime; chlorocyclizine; chloropyramine; chlorothen; chlorphenoxamine; cinnarizine; clemastine; clobenzepam; clobenztropine; clocinizine; cyclizine; deptropine; dexchlorpheniramine; dexchlorpheniramine maleate; diphenylpyraline; doxepin; ebastine; embramine; emedastine; epinastine; etymemazine hydrochloride; fexofenadine; histapyrrodine; hydroxyzine; isopromethazine; isothipendyl; levocabastine; mebhydroline; mequitazine; methafurylene; methapyrilene; metron; mizolastine; olapatadine; orphenadrine; phenindamine; pheniramine; phenyltoloxamine; p-methyldiphenhydramine; pyrrobutamine; setastine; talastine; terfenadine; thenyldiamine; thiazinamium; thonzylamine hydrochloride; tolpropamine; triprolidine; and tritoqualine.

Antihistamine analogs may also be used. Antihistamine analogs include 10-piperazinylpropylphenothiazine; 4-(3-(2-chlorophenothiazin-10-yl)propyl)-1-piperazineethanol dihydrochloride; 1-(10-(3-(4-methyl-1-piperazinyl)propyl)-10H-phenothiazin-2-yl)-(9CI) 1-propanone; 3-methoxycyproheptadine; 4-(3-(2-Chloro-10H-phenothiazin-10-yl)propyl)piperazine-1-ethanol hydrochloride; 10,11-dihydro-5-(3-(4-ethoxycarbonyl-4-phenylpiperidino)propylidene)-5H-dibenzo(a,d)cycloheptene; aceprometazine; acetophenazine; alimemazin (e.g., alimemazin hydrochloride); aminopromazine; benzimidazole; butaperazine; carfenazine; chlorfenethazine; chlormidazole; cinprazole; desmethylastemizole; desmethylcyproheptadine; diethazine (e.g., diethazine hydrochloride); ethopropazine (e.g., ethopropazine hydrochloride); 2-(p-bromophenyl-(p′-tolyl)methoxy)-N,N-dimethyl-ethylamine hydrochloride; N,N-dimethyl-2-(diphenylmethoxy)-ethylamine methylbromide; EX-10-542A; fenethazine; fuprazole; methyl 10-(3-(4-methyl-1-piperazinyl)propyl)phenothiazin-2-yl ketone; lerisetron; medrylamine; mesoridazine; methylpromazine; N-desmethylpromethazine; nilprazole; northioridazine; perphenazine (e.g., perphenazine enanthate); 10-(3-dimethylaminopropyl)-2-methylthio-phenothiazine; 4-(dibenzo(b,e)thiepin-6(11H)-ylidene)-1-methyl-piperidine hydrochloride; prochlorperazine; promazine; propiomazine (e.g., propiomazine hydrochloride); rotoxamine; rupatadine; Sch 37370; Sch 434; tecastemizole; thiazinamium; thiopropazate; thioridazine (e.g., thioridazine hydrochloride); and 3-(10,11-dihydro-5H-dibenzo(a,d)cyclohepten-5-ylidene)-tropane.

Other compounds that may be used in the present compositions include AD-0261; AHR-5333; alinastine; arpromidine; ATI-19000; bermastine; bilastin; Bron-12; carebastine; chlorphenamine; clofurenadine; corsym; DF-1105501; DF-11062; DF-1111301; EL-301; elbanizine; F-7946T; F-9505; HE-90481; HE-90512; hivenyl; HSR-609; icotidine; KAA-276; KY-234; lamiakast; LAS-36509; LAS-36674; levocetirizine; levoprotiline; metoclopramide; NIP-531; noberastine; oxatomide; PR-881-884A; quisultazine; rocastine; selenotifen; SK&F-94461; SODAS-HC; tagorizine; TAK-427; temelastine; UCB-34742; UCB-35440; VUF-K-8707; Wy-49051; and ZCR-2060.

Still other compounds that may be used in the present compositions are described in U.S. Pat. Nos. 3,956,296; 4,254,129; 4,254,130; 4,282,233; 4,283,408; 4,362,736; 4,394,508; 4,285,957; 4,285,958; 4,440,933; 4,510,309; 4,550,116; 4,692,456; 4,742,175; 4,833,138; 4,908,372; 5,204,249; 5,375,693; 5,578,610; 5,581,011; 5,589,487; 5,663,412; 5,994,549; 6,201,124; and 6,458,958.

The compositions that are applied to the site of the hair follicle may include an antimicrobial agent. Useful antimicrobial agents include, without limitation, benzyl benzoate, benzalkonium chloride, benzoic acid, benzyl alcohol, butylparaben, ethylparaben, methylparaben, propylparaben, camphorated metacresol, camphorated phenol, hexylresorcinol, methylbenzethonium chloride, cetrimide, chlorhexidine, chlorobutanol, chlorocresol, cresol, glycerin, imidurea, phenol, phenoxyethanol, phenylethylalcohol, phenylmercuric acetate, phenylmercuric borate, phenylmercuric nitrate, potassium sorbate, sodium benzoate, sodium proprionate, sorbic acid, and thiomersal.

The antimicrobial may be from about 0.05% to 0.5% by weight of the total composition, except for camphorated phenol and camphorated metacresol. For camphorated phenol, the preferred weight percentages are about 8% to 12% camphor and about 3% to 7% phenol. For camphorated metacresol, the preferred weight percentages are about 3% to 12% camphor and about 1% to 4% metacresol.

The compositions that are applied to the site of the hair follicle may include an anti-inflammatory agent. Useful antiinflammtory agents include, without limitation, Non-Steroidal Anti-Inflammatory Drugs (NSAIDs) (e.g., naproxen sodium, diclofenac sodium, diclofenac potassium, aspirin, sulindac, diflunisal, piroxicam, indomethacin, ibuprofen, nabumetone, choline magnesium trisalicylate, sodium salicylate, salicylsalicylic acid (salsalate), fenoprofen, flurbiprofen, ketoprofen, meclofenamate sodium, meloxicam, oxaprozin, sulindac, and tolmetin), COX-2 inhibitors (e.g., rofecoxib, celecoxib, valdecoxib, and lumiracoxib), and corticosteroids (e.g., alclometasone dipropionate, amcinonide, betamethasone dipropionate, betamethasone valerate, clobetasol propionate, desonide, desoximetasone, dexamethasone, diflorasone diacetate, flucinolone acetonide, flumethasone, fluocinonide, flurandrenolide, halcinonide, halobetasol propionate, hydrocortisone butyrate, hydrocortisone valerate, methylprednisolone, mometasone furoate, prednisolone, or triamcinolone acetonide).

The compositions that are applied to the site of the hair follicle may include a nonsteroidal immunosuppressant. Suitable immunosuppressants include cyclosporine, tacrolimus, rapamycin, everolimus, and pimecrolimus.

The cyclosporines are fungal metabolites that comprise a class of cyclic oligopeptides that act as immunosuppressants. Cyclosporine A is a hydrophobic cyclic polypeptide consisting of eleven amino acids. It binds and forms a complex with the intracellular receptor cyclophilin. The cyclosporine/cyclophilin complex binds to and inhibits calcineurin, a Ca²⁺-calmodulin-dependent serine-threonine-specific protein phosphatase. Calcineurin mediates signal transduction events required for T-cell activation (reviewed in Schreiber et al., Cell 70:365-368, 1991). Cyclosporines and their functional and structural analogs suppress the T cell-dependent immune response by inhibiting antigen-triggered signal transduction. This inhibition decreases the expression of proinflammatory cytokines, such as IL-2. Many different cyclosporines (e.g., cyclosporine A, B, C, D, E, F, G, H, and I) are produced by fungi. Cyclosporine A is a commercially available under the trade name NEORAL from Novartis. Cyclosporine A structural and functional analogs include cyclosporines having one or more fluorinated amino acids (described, e.g., in U.S. Pat. No. 5,227,467); cyclosporines having modified amino acids (described, e.g., in U.S. Pat. Nos. 5,122,511 and 4,798,823); and deuterated cyclosporines, such as ISAtx247 (described in U.S. Patent Application Publication No. 2002/0132763 A1). Additional cyclosporine analogs are described in U.S. Pat. Nos. 6,136,357, 4,384,996, 5,284,826, and 5,709,797. Cyclosporine analogs include, but are not limited to, D-Sar (α-SMe)³ Val²-DH-Cs (209-825), Allo-Thr-2-Cs, Norvaline-2-Cs, D-Ala(3-acetylamino)-8-Cs, Thr-2-Cs, and D-MeSer-3-Cs, D-Ser(O—CH₂CH₂—OH)-8-Cs, and D-Ser-8-Cs, which are described in Cruz et al., Antimicrob. Agents Chemother. 44:143 (2000).

Tacrolimus and tacrolimus analogs are described by Tanaka et al. (J. Am. Chem. Soc., 109:5031 (1987)) and in U.S. Pat. Nos. 4,894,366, 4,929,611, and 4,956,352. FK506-related compounds, including FR-900520, FR-900523, and FR-900525, are described in U.S. Pat. No. 5,254,562; O-aryl, O-alkyl, O-alkenyl, and O-alkynylmacrolides are described in U.S. Pat. Nos. 5,250,678, 532,248, 5,693,648; amino O-aryl macrolides are described in U.S. Patent No. 5,262,533; alkylidene macrolides are described in U.S. Pat. No. 5,284,840; N-heteroaryl, N-alkylheteroaryl, N-alkenylheteroaryl, and N-alkynylheteroaryl macrolides are described in U.S. Pat. No. 5,208,241; aminomacrolides and derivatives thereof are described in U.S. Pat. No. 5,208,228; fluoromacrolides are described in U.S. Pat. No. 5,189,042; amino O-alkyl, O-alkenyl, and O-alkynylmacrolides are described in U.S. Pat. No. 5,162,334; and halomacrolides are described in U.S. Pat. No. 5,143,918.

Tacrolimus is extensively metabolized by the mixed-function oxidase system, in particular, by the cytochrome P-450 system. The primary mechanism of metabolism is demethylation and hydroxylation. While various tacrolimus metabolites are likely to exhibit immunosuppressive biological activity, the 13-demethyl metabolite is reported to have the same activity as tacrolimus.

Pimecrolimus is the 33-epi-chloro derivative of the macrolactam ascomyin. Pimecrolimus structural and functional analogs are described in U.S. Pat. No. 6,384,073.

Rapamycin structural and functional analogs include mono- and diacylated rapamycin derivatives (U.S. Pat. No. 4,316,885); rapamycin water-soluble prodrugs (U.S. Pat. No. 4,650,803); carboxylic acid esters (PCT Publication No. WO 92/05179); carbamates (U.S. Pat. No. 5,118,678); amide esters (U.S. Pat. No. 5,118,678); biotin esters (U.S. Pat. No. 5,504,091); fluorinated esters (U.S. Pat. No. 5,100,883); acetals (U.S. Pat. No. 5,151,413); silyl ethers (U.S. Pat. No. 5,120,842); bicyclic derivatives (U.S. Pat. No. 5,120,725); rapamycin dimers (U.S. Pat. No. 5,120,727); O-aryl, O-alkyl, O-alkyenyl and O-alkynyl derivatives (U.S. Pat. No. 5,258,389); and deuterated rapamycin (U.S. Pat. No. 6,503,921). Additional rapamycin analogs are described in U.S. Pat. Nos. 5,202,332 and 5,169,851.

The compositions that are applied to the site of the hair follicle may include a retinoid. Useful retinoids include, without limitation, 13-cis-retinoic acid, 9-cis retinoic acid, all-trans-retinoic acid, etretinate, acitretin, retinol, retinal, tretinoin, alitretinoin, isotretinoin, tazarotene, bexarotene, and adapelene.

In certain embodiments, the compositions that are applied to the site of the hair follicle may include a channel opener. Useful channel openers include, without limitation, minoxidil, diazoxide, and phenyloin.

In other embodiments, an anti-androgen can be used in the compositions that are applied to the site of the hair follicle. Useful anti-androgens include, without limitation, finasteride, flutamide, diazoxide, 11alpha-hydroxyprogesterone, ketoconazole, RU58841, dutasteride, fluridil, QLT-7704, and anti-androgen oligonucleotides.

In certain embodiments, the compositions that are applied to the site of the hair follicle may include an antibiotic. Useful antibiotics include, without limitation, penicillin G, penicillin V, methicillin, oxacillin, cloxacillin, dicloxacillin, nafcillin, ampicillin, amoxicillin, carbenicillin, ticarcillin, mezlocillin, piperacillin, azlocillin, temocillin, cepalothin, cephapirin, cephradine, cephaloridine, cefazolin, cefamandole, cefuroxime, cephalexin, cefprozil, cefaclor, loracarbef, cefoxitin, cefmatozole, cefotaxime, ceftizoxime, ceftriaxone, cefoperazone, ceftazidime, cefixime, cefpodoxime, ceftibuten, cefdinir, cefpirome, cefepime, BAL5788, BAL9141, imipenem, ertapenem, meropenem, astreonam, clavulanate, sulbactam, tazobactam, streptomycin, neomycin, kanamycin, paromycin, gentamicin, tobramycin, amikacin, netilmicin, spectinomycin, sisomicin, dibekalin, isepamicin, tetracycline, chlortetracycline, demeclocycline, minocycline, oxytetracycline, methacycline, doxycycline, erythromycin, azithromycin, clarithromycin, telithromycin, ABT-773, lincomycin, clindamycin, vancomycin, oritavancin, dalbavancin, teicoplanin, quinupristin and dalfopristin, sulphanilamide, para-aminobenzoic acid, sulfadiazine, sulfisoxazole, sulfamethoxazole, sulfathalidine, linezolid, nalidixic acid, oxolinic acid, norfloxacin, perfloxacin, enoxacin, ofloxacin, ciprofloxacin, temafloxacin, lomefloxacin, fleroxacin, grepafloxacin, sparfloxacin, trovafloxacin, clinafloxacin, gatifloxacin, moxifloxacin, gemifloxacin, sitafloxacin, metronidazole, daptomycin, garenoxacin, ramoplanin, faropenem, polymyxin, tigecycline, AZD2563, and trimethoprim.

Growth factors and growth factor antagonists can also be used in the compositions that are applied to the site of the hair follicle.

The composition may comprise an active ingredient for stimulating hair growth. Nonlimiting examples include monoxidil, finasteride, dutasteride, a copper peptide, saw palmetto extract, black cohosh, caffeine, or any combination thereof.

The composition that is applied to the site of the hair follicle may comprise a biological material. For example, DNA, RNA, cells (such as stem cells, nurse cells, keratinocytes), cellular components (collagen, elastin, cytoskeletal components, keratin), proteins, skin graft material, antibodies, viruses, or any other living or quasi-living material or product of a living system. As described more fully below, the composition, whether a biological material or another type of material may be applied substantially directly to the site of the hair follicle, and may even be applied substantially into a channel or other cavity formed therethrough.

The composition may comprise protective covering or sealant. Polymers, skin grafts, synthetic skin, biological glues, or any other material that is capable of forming a protective layer or seal at the site of the hair follicle (e.g., over the portion of the body surface at which the site of the hair follicle is located) is contemplated. In certain embodiments, the application of a composition to the site of the hair follicle may include the application of a material or compound of any other type described herein, sequentially followed by the application of a protective covering or sealant.

A biocompatible, synthetic skin substitute may be placed on a portion of tissue that has been injured in accordance with the present disclosure, especially if the wound is deep, covers large area, and has been bulk ablated. This process can help minimize or prevent the rapid wound contraction that occurs after loss of a large area of tissue, frequently culminating in scar tissue formation and loss of skin function. The biocompatible synthetic skin substitute may be impregnated with depots of slow releasing stem cell signaling molecules to channel the proliferating stem cell population toward hair follicle germ formation. This method of treatment may enable treating a large bald area on the scalp in one session at the treatment clinic. Other molecules may be co-eluted at the site through the skin substitute, such as anesthetics and antibiotics, to prevent further pain and minimization of infection. The skin substitute containing drug, as described herein, may also be pre-cooled and applied to the wound to provide a feeling of comfort to the patient. This mode of drug application may prevent the drug from being cleared away from the wound site, as the wound heals.

It is also envisioned that a compound absorbing light at specific wavelengths (e.g., between 1000-1600 nm may be included in a composition according to the present disclosure for the purpose of efficient channeling of light to heat energy. This method of channeling energy may cause micro-zones of thermal injury within the body surface. The compound may be delivered to the body surface homogenously in the treatment zone, then subsequently irradiated, for example, with a non-ablative laser, to efficiently capture the vibrational energy of the beam. This method may result in evenly distributed and deep thermal injury, without causing tissue vaporization.

Any other material or compound that may be useful for promoting or aiding in a desired outcome, including regeneration, remodeling, resurfacing, restoration, follicular neogenesis, neocollagenesis, stem cell recruitment, activation, or differentiation, reepitheliazation, wound healing, or any other desired biological or mechanical modification, may be applied to the site of the hair follicle in accordance with the present disclosure. Other suitable materials are described in WO/2008/143928, which is incorporated herein by reference in its entirety. Other materials of interest may include pigments, inks, dyes, or toxins (including neurotoxins, such as botulinum toxin).

The composition may be applied as a fluid (e.g., a liquid, gel, or gas) or as a solid (e.g., as a particulate material). The composition may be applied to the body surface or to some location beneath the body surface (e.g., into the tissue beneath the surface). The propulsion of drug-containing particles into a body surface—in particular, skin—is described at length PCT/US08/11979, the contents of which are incorporated herein in their entirety. The composition may comprise components that cause gelling or hardening of the composition. The gelling or hardening may occur as a result of a reaction between two or more components within the composition (as discussed more fully herein, in such embodiments the application of the composition may include the mixing of reactive components that form a gel following application of the composition to the target area). Exemplary compositions that form gels are disclosed infra. In other embodiments, the composition may be accelerated and “shot” in a narrow stream into part or all of the site of the hair follicle, much in the manner of transdermal particle injection systems or “gene guns” that are used to deliver a narrow stream of material through the stratum corneum layer of skin.

Compositions for topical administration for preferably local but also possible systemic effect, include emulsions, solutions, suspensions, creams, gels, hydrogels, ointments, dusting powders, dressings, elixirs, lotions, suspensions, tinctures, pastes, powders, crystals, foams, films, aerosols, irrigations, sprays, suppositories, sticks, bars, ointments, bandages, wound dressings, microdermabrasion or dermabrasion particles, drops, and transdermal or dermal patches. The topical formulations can also comprise micro- and nano-sized capsules, liposomes, micelles, microspheres, microparticles, nanosystems, e.g., nanoparticles, nano-coacervates and mixtures thereof. See, e.g., International Patent Application Publication Nos. WO 2005/107710, published Nov. 17, 2005, and WO 2005/020940, published Mar. 10, 2005, each of which is incorporated herein by reference in its entirety. In one embodiment, the nano-sized delivery matrix is fabricated through a well-defined process, such as a process to produce lithium encapsulated in a polymer. In another embodiment, a drug-releasing compound is spontaneously assembled in aqueous solutions, such as in liposomes and micelles.

The modality for segmenting a follicle may also be used to apply the composition to the site of the hair follicle. For example, a needle may be used to segment a hair follicle and as a composition-delivery conduit. The propulsion of drug-containing particles into a body surface invokes a microdermabrasion model to segment a follicle area while simultaneously delivering a drug-containing composition (see PCT/US08/11979). A high-pressure jet of fluid (with or without abrasive particles within the fluid) may be used to segment a follicle, and if the fluid contains a composition, then segmentation and application of a composition may be performed contemporaneously. Water jet technology, for example, was developed in the 1950's and may be used to cut or puncture soft or hard materials (see, for example, Flow International Corporation, Kent, Wash.). Any other approach for using the segmenting modality for applying a composition to the site of a hair follicle may be used.

The composition that is applied to the site of a hair follicle may allow for the delivery of physiologically active material to the site of a hair follicle immediately or after a period of delay. For example, the composition may comprise a physiologically active compound that will contact the site of a hair follicle as soon as the composition is applied and/or may comprise a physiologically active compound that is encapsulated within a degradable material so that the compound does not contact the site of a hair follicle until the degradable material breaks down or is worn away in situ. In this and other embodiments, the period of delay may be minutes, hours, or days, for example, about 10 minutes, about 30 minutes, about one hour, about two hours, about three hours, about six hours, about eight hours, about 12 hours, about 24 hours, about 36 hours, about two days, about three days, about one week, about two weeks, about three weeks, or any other desired period of delay. Once delivery of the physiologically active material has commenced, the rate of release may have any desired profile, such as constant or ascending. Those of ordinary skill in the pharmaceutical arts will readily appreciate available methods for achieving a desired release profile. For example, a plurality of tiny “pills” that individually comprise a dose of a drug and a wall may be included in the composition that is delivered to the site of a hair follicle, wherein the plurality of tiny pills comprises at least two separate populations of pills, wherein the respective walls of the pills in the first population are thicker than the respective walls of the pills in the second population, and wherein the respective doses of drug within the pills in the first population are greater than the respective doses of drug within the pills in the second population in order to provide for an increasing release rate. Procedures for manufacturing tiny pills are disclosed in U.S. Pat. Nos. 4,434,153; 4,721,613; 4,853,229; 2,996,431; 3,139,383 and 4,752,470.

The preparation of various pharmaceutical formulations and exemplary components thereof, including controlled and extended release formulations, topical formulations, emulsifying excipients for use in formulations, gelling agents, hydrocolloids, cross-linking agents, and plasticizers are disclosed in WO 2008/143928, the entire contents of which are incorporated herein by reference.

Any gel or other matrix may be used pursuant to the present compositions. Gels or other matrices that optionally comprise one or more physiologically active compounds may be delivered into “micro”-channels (hereafter, “channels”) created by such modalities as fractional lasers, microneedle flat arrays or rollers, or any other device that creates channels in the body surface. For example, when the body surface is skin, the channel may extend through the stratum corneum, epidermis, and partially or fully into the dermis.

The matrices may be delivered as a drug-containing liquid into the channels, for example, by a device that can deliver precise volumes. In addition to the drug, the liquid, or the “vehicle” may contain a polymer, or a combination of polymers that either are thermoreversible, or viscosity enhancing, or act as ionic supports for the drug. By definition, “thermoreversible” means that aqueous solutions of the polymer display viscoelastic properties that are “reversed” or opposite to what is typically observed in fluids when they are heated or cooled. As an example, aqueous solutions of Polyethylene oxide-co-polypropylene oxide-co-polyethylene oxide (PEO-PPO-PEO) polymers have very low viscosity when cooled, slowly forming a hydrogel when warmed up to physiological temperatures. This property can be modulated by varying the concentration of the polymer and/or varying the ratio of the PEO/PPO segments. Thus, the temperature at which the polymer in solution reaches gelation is lower when the concentration of the polymer is higher. In an application of this property to current embodiment, a cold low viscosity solution can be “streamed” into the channels, which would then form a physically crosslinked gel upon warming to body temperature. By definition, a “physical cross-link” is not a covalent link, but is based on hydrogen bonds, ionic interactions and molecular entanglement of polymer chains. Delivery of a cold solution also provides a comfortable or soothing “feel” to the patient. A physically crosslinked solution is not a permanent crosslink, and generally diffuses or clears from the site by absorption. These types of polymer vehicles are preferred over permanently crosslinked polymers or hydrogels due to their biocompatibility with surrounding cells and tissues. Permanently crosslinked gels are biocompatible only if they are bioabsorbable by hydrolysis or proteolysis.

The polymer matrix that is delivered into the channels may comprise a biodegradable polymer than is degradable by hydrolysis or proteolysis. In addition, the biodegradable polymer may have difunctional crosslinkable groups that react to form covalent crosslinks in order to form a hydrogel. Hydrogel formation can be through use of redox reactive groups, or photoreactive groups or crosslinking through reaction between a highly reactive electrophile and nucleophile. For this embodiment, crosslinking initiators need to be part of the matrix. Crosslinking by polymerization can be initiated by a redox initiator, or a photoinitiator. UV light, visible light or infrared can be used to initiate the crosslinking reaction to form the hydrogel. In one embodiment, a laser or other form of electromagnetic energy used to create the channels can be used to crosslink the hydrogel.

The “biodegradable polymer” disclosed above may contain water-soluble moieties such as polyethylene oxide, chain extended by lactates, glycolates and end-capped with crosslinkable moieties such as acrylates. The biodegradable polymer may be thermoreversible, wherein the polymer is highly fluid when cold and viscous at higher temperatures, but is biodegradable and crosslinkable. An example of this type of polymer is PEO-PPO-PEO. In another embodiment, the crosslink density or mesh size of the hydrogel can be modulated by using polymers of varying functionalities. For example, a four-armed polymer core can be used to achieve a hydrogel with a smaller mesh size than one achieved with a difunctional polymer core.

In another embodiment of a crosslinkable, biodegradable hydrogel, a biopolymer that reacts with components in tissue can be used to form a hydrogel.

Physiologically active compounds that are contained within physically crosslinked gels as described above are released from the matrix. The rate of release from this matrix is primarily controlled by the properties of the drug, i.e., if the molecular weight of the drug is much less than the pore size of the matrix. Typically, this is the case for small molecule drugs, with release rates being governed by the drug's solubility in water. A hydrophobic drug can be incorporated into an aqueous gel as microparticulate drug, with its release from the matrix rate-limited by the rate of dissolution of the drug in water. A hydrophilic drug, if not bound to the matrix by an interaction such as an ionic interaction, would be released from a physically crosslinked matrix very quickly, depending upon the molecular weight of the drug. For example, this type of matrix would be more appropriate for a hydrophilic protein than a hydrophilic small molecule. To slow down release of an ionic hydrophilic drug, use of a matrix that can ionically bind the drug, is a favorable option. Additionally, the hydrophilic drug such as a lithium salt, can be incorporated into solid lipid nanoparticles, then suspended in a viscous liquid like a cream, gel or emulsion.

Drugs that are small molecular and hydrophilic may be encapsulated into biodegradable microspheres, and then incorporated into a gel for delivery into a channel. This method can significantly slow down the diffusion of the drug from the site. The rate of release of the drug from the microspheres can be modulated by choice of the polymer. For example, a PLG polymer of molecular weight 12,000 Daltons releases drug at a much slower rate than a PLG polymer of molecular weight 30,000 Daltons. In another example, a PLG polymer with acid end groups release drug at faster rate than a PLG polymer with ester end groups. In another example, polylactic acid (PLA) releases drug very slowly, due to its low rate of hydrolytic degradation. Thus, the rate of drug release can be modulated appropriately by choice of the polymer used to encapsulate the drug. This approach can be used in a similar fashion for hydrophobic drugs.

In some embodiments, a drug-containing polymer solution is delivered into the channels using a delivery device and the solvent used to dissolve the biodegradable polymer diffuses out into surrounding tissue, leaving behind substantially solid columns of drug-containing matrix. An example of this type of matrix is PLG polymer+drug dissolved in a low molecular weight polyethylene glycol (PEG 300) as the solution to be delivered into the channels. After administration, the water soluble PEG300 diffuses into the surrounding tissue, leaving behind what is effectively a sustained release drug delivery system.

In another embodiment, the drug is encapsulated in a cavitrant molecule such as cyclodextrin, and derivatives thereof.

The application of the composition to the site of the hair follicle may be accomplished by any method that contacts the composition with the site of the hair follicle. For example, the composition may be sprayed, dripped, painted, propelled, misted, or injected in order to apply it to the site of the hair follicle. The application of the composition to the site of the hair follicle may be topical, may be to some location at the site of the hair follicle that is interior to the body surface, or both. In some embodiments, the composition is a fluid that is sprayed onto the site of the hair follicle. In other embodiments, the composition is sprayed, propelled, or injected into the site of a hair follicle that has been segmented, which may include contacting only the injured portion of the site of the hair follicle with the composition, contacting only the site of the hair follicle with the composition, contacting substantially only the site of the hair follicle with the composition (i.e., wherein only incidental amounts of composition are applied to areas of the body surface beyond the site of the hair follicle), or contacting site of the hair follicle and one or more adjacent areas of the body surface with the composition.

When the site of the hair follicle is injured by removing a column of tissue to form a channel, the composition may be applied substantially directly into the channel. The application of the composition “substantially directly” into the channel refers to the delivery of one or more aliquots of composition into the channel that may or may not include the delivery of an amount of composition to the site of the hair follicle outside of the channel, to one or more adjacent area of the body surface, or both. Depending on the chosen means for applying the composition substantially directly into the channel, the composition may be precisely delivered into the channel with no or only incidental amounts of composition being delivered outside of the channel. For example, inkjet-type technology may be used for precise application of the composition into the channel, and in this manner, a composition containing a physiologically active compound, a biological material, or any other desired agent may be introduced into the body surface at a desired location. The delivery of cells via inkjet printer has been reported (see, e.g., S. Webb, “Life in Print. Cell by cell, ink-jet printing builds living tissues”. Science News, Vol. 173, Jan. 26, 2008), and such technology may be used for the precise administration of biological material, physiologically active compound, or the like into a incision or channel in a site of the hair follicle in accordance with the present disclosure. In some embodiments, the composition that is applied substantially directly into a channel at the site of the hair follicle may be a fluid that forms a gel in situ. A composition of this variety may release a physiologically active compound into the site of the hair follicle at a desired release rate, e.g., an immediate release or a controlled rate of release over time. FIG. 3 illustrates (a) the use of a fractional laser to form a hole in human skin, after which (b) the hole is filled with a highly viscous drug-containing gel via an ink-jet precision fill device. At step (c), body heat or other external factors crosslink the gel into a stable drug-releasing matrix, and (d) drug is released from the matrix over time. Not depicted in FIG. 3 is the fact that the fractional laser (and therefore the hole that is formed thereby) will transect a follicle in order to segment the follicle into two or more disunited subunits. Furthermore, the fractional laser may be translated relative to the body surface in order to form a slice- or slit-type injury into which a composition may be delivered in accordance with the preceding.

Thus, a drug containing gel matrix can be delivered into the holes created by what is tantamount to a fractional FTE modality (e.g., laser, micro needles, miniature punch biopsy needles, and the like). Poly-phasic biocompatible gels such as pluronic “F-127” can be produced in a highly viscous drug contacting solution or emulsion. At room temperature, these solutions can be readily delivered via ink jet or by precision industrial “micro-fill” technology. MicroFab, Inc. of Plano, Tex. provides a piezo-based high-speed fluidic delivery systems that can accurately deliver these volumes (e.g., ⅓ mm³ per hole). Once the drug contacting pluronic solution is delivered into the hole, body heat permanently changes the highly viscous solution into a stable gel. The gel may then release drug over time as the holes heal. In accordance with the present disclosure, drug may be released over about 12 hours to about 20 days, about 1 day to about 10 days, or about 3 days to about 7 days, or over other longer or shorter periods of time, as desired. Other highly viscous drug contacting macromonomeric biocompatible solutions (examples described supra) can be cross-linked into a stable drug releasing hydrogel. For cross-linking to occur, the polymer must have crosslinkable moieties such as acrylates. Crosslinking can be achieved by incorporating a photoinitiator such as Darocure or Irgacure and initiated by light (UV light, visible light, laser light). Crosslinking can also be achieved using a GRAS redox initiator, wherein the crosslinking mechanism does not involve heat, or light, but an oxidation reduction reaction.

The step of applying “a composition” to the site of the hair follicle may include the application of two or more compositions, and the compositions may respectively be applied using a desired modality. For example, a first composition may be applied to the site of the hair follicle in the form of a fluid that is applied substantially directly into a channel that was formed at the site of the hair follicle, and a second composition may be a protective covering or seal that is applied onto the site of the hair follicle and over the injury to protect or seal the first composition within the channel or otherwise shield the injury from the ambient environment. In such instances, the first composition may be applied using inkjet-type technology, and the second composition may be applied using conventional spray technology. All combinations of composition types and application modalities are contemplated as being embraced by the present disclosure.

The segmentation of the first hair follicle and optional application of a composition may be followed by the identification of a further hair follicle, the segmentation of the further hair follicle into at least two disunited subunits, and the optional application of the same or a different composition to the site of the further hair follicle. The “identification” of a further hair follicle may be in accordance with any one or more of the procedures or mechanisms described above with respect to the identification of the first hair follicle. For example, the identification of a further hair follicle may be accomplished by locating a hair that corresponds to a subsurface follicle (i.e., an indirect assessment of the location of a follicle), or by direct location of a follicle; may comprise an assessment of certain characteristics of that follicle, such as its size, depth, and angle relative to the body surface; may comprise an assessment of general characteristics of the body surface; may be accomplished using any appropriate device or other modality; and the like.

The “identification” of a further hair follicle may comprise the selection of a new target area on the body surface having a preselected geometry relative to the first hair follicle. The “preselected geometry” may be based on a set of coordinates that collectively form a pattern, wherein the first hair follicle and the new target area respectively represent successive coordinates within the pattern. For example, the pattern from which the preselected geometry is derived may be based upon a rectilinear grid, a curvilinear grid, a tessellation, a Fibonacci sequence, or any other regular, semiregular, or irregular arrangement of coordinates (points) or shapes. Thus, the first hair follicle may represent a first coordinate or shape within the pattern, and the new target area will constitute the succeeding coordinate or shape with the same pattern. The “preselected geometry” need not be selected from an ordered array of coordinates or shapes, and the further target area may in fact be assigned through a randomized selection; in such instances, the first hair follicle may represent a first coordinate or shape, and the new target area will constitute a second coordinate having a spatial relationship relative to the first hair follicle that is randomly assigned, i.e., is “predetermined” in the sense that it was known beforehand that its spatial relationship to the first hair follicle would be randomly assigned.

The selection of the new target area may be performed by a human controller, or may be performed by computerized system having the appropriate software. A human controller may provide initial instructions to a computer in order to identify a particular pattern or other basis for the preselected geometry (for example, the human controller may select a rectilinear grid as the pattern upon which the determination of the new target area or areas is based), and a computerized system may select the new target areas by proceeding in accordance with the initial instructions that were provided by the human controller. Thus, the computerized system and software may be capable of proceeding according to any of a number of different preloaded patterns, and may only require the input of a human controller as to which pattern should be used in order to commence the selection of a new target area or areas. One of ordinary skill in the art will readily appreciate how to obtain or create software that includes the instructions necessary for selecting one or more new target areas based on an ordered array or in accordance with a randomized selection.

The segmentation of the further hair follicle and the application of a same or different composition may respectively be performed in accordance with any of the any one or more of the procedures or mechanisms described above with respect to the first hair follicle. In addition, the identification of a further hair follicle, segmentation of the further hair follicle into at least two disunited subunits, and the optional application of the same or different composition to the site of the further hair follicle may be performed iteratively to identify, segment, and optionally apply a composition to one or more additional hair follicles. In this manner, each member or a desired subset of a population of hair follicles that are present on a body surface may be identified and undergo segmentation in accordance with the present disclosure. The iterative segmentation of hair follicles on a body surface may increase the number of hair-producing follicles on the body surface by two-fold or more, representing a valuable treatment for subjects suffering from male- or female-pattern hair loss, pathological hair loss, or hair loss after injury. The present methods and systems are able to maximize hair growth in and near areas where follicles exist but are too few in number to provide hair at a desired density, including the scalp, the face, and the margins of wounds and scars. For example, if the body surface comprises a population of hair follicles at the margins of a patch of scar tissue, the population of hair follicles may be subjected to the iterative treatment (i.e., identification, segmentation, and optionally exposure to a composition) in accordance with the present disclosure.

FIG. 4 depicts how the segmentation of a hair follicle at the margin of scar tissue can be used to generate new hair follicles for producing hair that grows into the scar tissue, thereby providing beneficial cosmetic results. In FIG. 4A, an incisor 12 is used to segment hair follicle 6 at the margin of scar tissue 14 into upper 6a and lower 6b portions. As shown in FIG. 4B, the shifting of tissue due to lines of tension in the body surface (arrow T) causes the upper portion 6a and lower portion 6b to shift relative to one another, leading to two spatially disunited segments of follicle 6. Because each of portions 6a and 6b contain a stem cell reservoir that is capable of driving the formation of an entire hair (see Toscani M, et al., Dermatol Surg 2009; 35:1119-1125), a complete new follicle and hair grows from each of portions 6a and 6b, and the new hair and follicle 16 from lower portion 6b grows into scar tissue 14 beyond the transitional margin between the scar tissue and the normal tissue, thereby reducing the appearance of the scar tissue (i.e., by concealing it with naturally grown hair). Such process may be performed iteratively with respect to other hairs at the margins of the scar tissue, may be repeated with respect to new hair and follicle 16, or both, in order to further reduce the appearance of the scar tissue.

Alternatively, the segmentation of the first hair follicle and optional application of a composition may be performed contemporaneously with the segmenting of each of one or more further hair follicles into at least two disunited subunits. In addition, the same or a different composition as that which is applied to the site of the first hair follicle may be applied to the site of the further hair follicle contemporaneously with the application of a composition to the site of the first hair follicle. Like the step of applying a composition to the first hair follicle, the application of a composition to the further hair follicle is optional, and may be (but need not) be performed contemporaneously with the application of the composition to the first hair follicle. The application of a composition to a further hair follicle may be performed or omitted independently from whether or not a composition is applied to the first hair follicle. The segmentation of the further hair follicle and the optional application of a same or different composition may respectively be performed in accordance with any of the any one or more of the procedures or mechanisms described above with respect to the first hair follicle. In certain embodiments, a single incision unit may be used to segment the first hair follicle contemporaneously with one or more further hair follicles. Incision units for such purposes and others, as well as exemplary processes of segmenting one or more further hair follicles contemporaneously with the segmentation of a first hair follicle are disclosed more fully infra.

In another aspect, systems for stimulating hair growth at a body surface are provided comprising an incision unit that is configured for applying a first incisor at an oblique angle relative to the body surface at the location of the first hair follicle for segmenting the first hair follicle into at least two disunited subunits.

Unless otherwise specified, any of the attributes, components, materials, or steps that are described with respect to one embodiment of the present disclosure (such as the disclosed methods) may be applicable to the attributes, components, materials, or steps of other embodiments of the present disclosure (including the disclosed systems).

The incision unit is configured for applying a first incisor at an oblique angle relative to the body surface at the location of the first hair follicle in order to segment the first hair follicle into at least two disunited subunits. Modalities for use as incisors for segmenting a hair follicle are disclosed above in connection with the present methods. The incision unit may be any appropriate mechanism for one or more of activating, deactivating, adjusting, housing, driving, and positioning the incisor. For example, the incision unit for a laser incisor may comprise the housing for the laser, a mechanism for positioning the laser (e.g., relative to the body surface and at an appropriate oblique angle), the circuitry for activating and deactivating the laser and for adjusting its power, and the like.

The incision unit may be configured for applying two or more incisors contemporaneously to the body surface, wherein each of the two or more incisors is applied at an oblique angle relative to the body surface. For example, the incision unit may be configured for partitioning a source laser beam into at least a first laser incisor and a second laser incisor, and applying both of the first laser incisor and the second laser incisor at an oblique angle relative to the body surface, wherein the first laser incisor and the second laser incisor are applied to the body surface contemporaneously. The partitioning of a source laser into at least two separate laser incisors may be accomplished by any suitable method, including the use of one or more prisms, one or more mirrors, one or more piezoelectric elements, or any other suitable mechanism. Routineers in the art will readily appreciate various techniques for partitioning a source laser beam into at least two separate laser incisors that are individually capable of segmenting a hair follicle into at least two disunited subunits, and any of such techniques are contemplated herein. In another embodiment, the incision unit may be configured for applying each of at least two cutting implements, for example needles or blades, at an oblique angle relative to the body surface, wherein at least a first cutting implement and a second cutting implement are applied to the body surface contemporaneously. The application of incisors to a body surface at an oblique angle is described supra, and such description fully applies to the present systems.

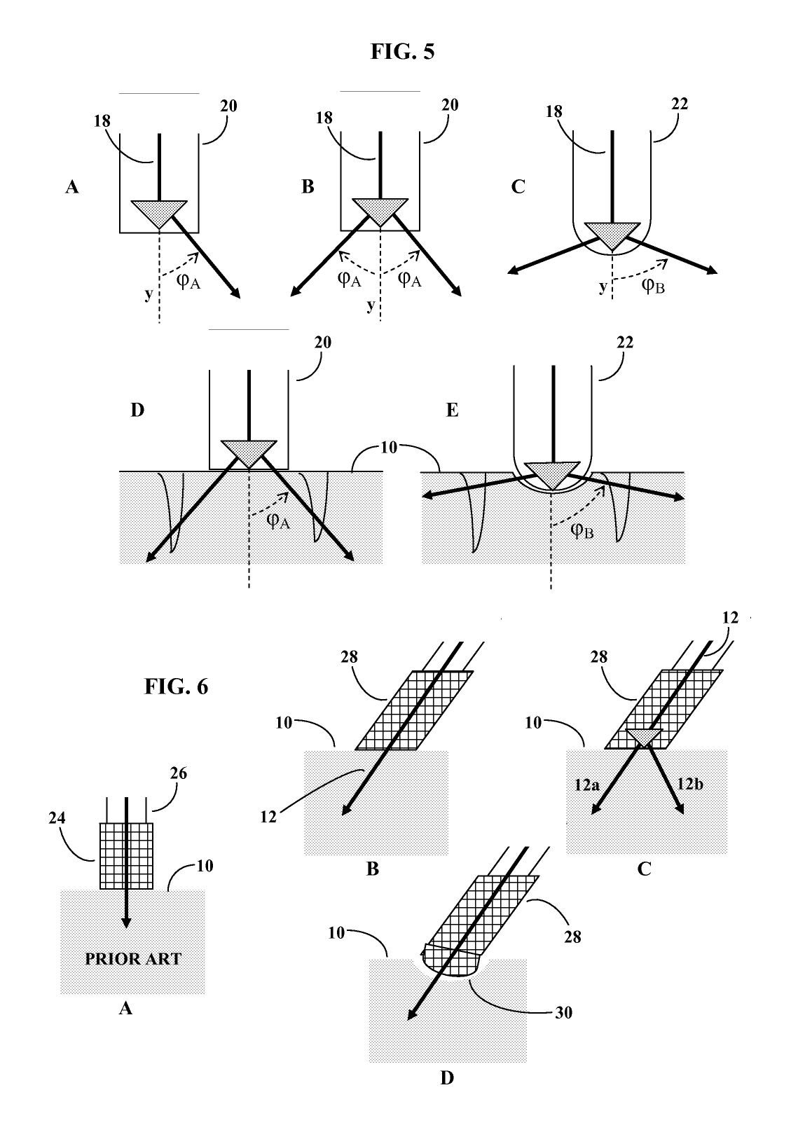

Exemplary incision units for applying a laser incisor to a body surface are depicted in FIG. 5. Piezoelectric elements, prisms, mirrors, or other means may be used for refracting a source laser beam 18 so that it is directed at an angle φ_{A} from the axis y that is perpendicular to the body surface (FIGS. 5A & 5D). Prisms, mirrors, or other means may also be used to split source laser beam 18 into at least two laser incisors that are each directed at an angle φ_{A} from the axis y that is perpendicular to the body surface 10 (FIGS. 5B & 5D). The incision unit may feature a substantially square housing 20 (FIGS. 5A, 5B, 5D), or may use a substantially rounded housing 22 to direct laser incisors at an angle φ_{B} from the axis y that is perpendicular to the body surface 10 (FIGS. 5C & 5E). A rounded housing 22 allows the incision unit to be pressed somewhat more deeply into the body surface 10, which may be useful for segmenting follicles that are located in the portion of the body surface 10 that is raised above the lowest point of the tip of the incision unit 22 by directing laser incisors at an angle φ_{B} from the axis y that is perpendicular to the body surface 10 (FIG. 5E).

As shown in FIG. 6A, conventional laser units (e.g., fractional laser units) employ a cubical or rectangular prismatic “cage” 24 that separates the laser source 26 from the body surface 10 and may include rollers to track the translation of the laser unit over the body surface. It has presently been discovered that the use of a novel cage design having a substantially rhombohedron-shaped configuration (FIGS. 6B-D) may be used to deliver laser beams at an angle that is not perpendicular to the body surface. FIG. 6B shows an exemplary rhombohedron-shaped cage 28 for use in delivering a laser beam incisor 12 at an oblique angle relative to a body surface 10. In FIG. 6C, the incision unit is equipped with a beam splitter, which may be a prism, mirrors, or piezoelectric element for partitioning a source laser beam 12 into a first laser incisor 12a and a second laser incisor 12b that are each directed at an oblique angle relative to the body surface 10. FIG. 6D depicts an exemplary rhobohedron-shaped cage 28 having a rounded tip 30 that can be pressed somewhat more deeply into the body surface 10, which may be useful for segmenting follicles that are located in the portion of the body surface 10 that is raised above the lowest point of the tip of the cage.

The incision unit may be configured for applying a single incisor or may be configured for applying two, three or more incisors. The incision unit may comprise a row or a regular or irregular array of incisors. For simplicity, embodiments comprising a row or array of source lasers are said to comprise a row or array of incision units, each of which house the respective source lasers, even though the unitary structure (the row or array of incision units) may also be referred to as an “incision unit”. A “row” may comprise two or more incision units, and an “array” may comprise three or more incision units. As described above, a laser incision unit may be configured for partitioning a source laser into two or more laser incisors, and in the present systems, the incision unit may comprise a row or array of source lasers of which some or all are each partitioned into two or more laser incisors. The present systems may comprise wheel-mounted rows or a wheel mounted arrays of needles or blades, and therefore incision units comprising multiple incisors are not limited to incision units that are configured for applying laser incisors.

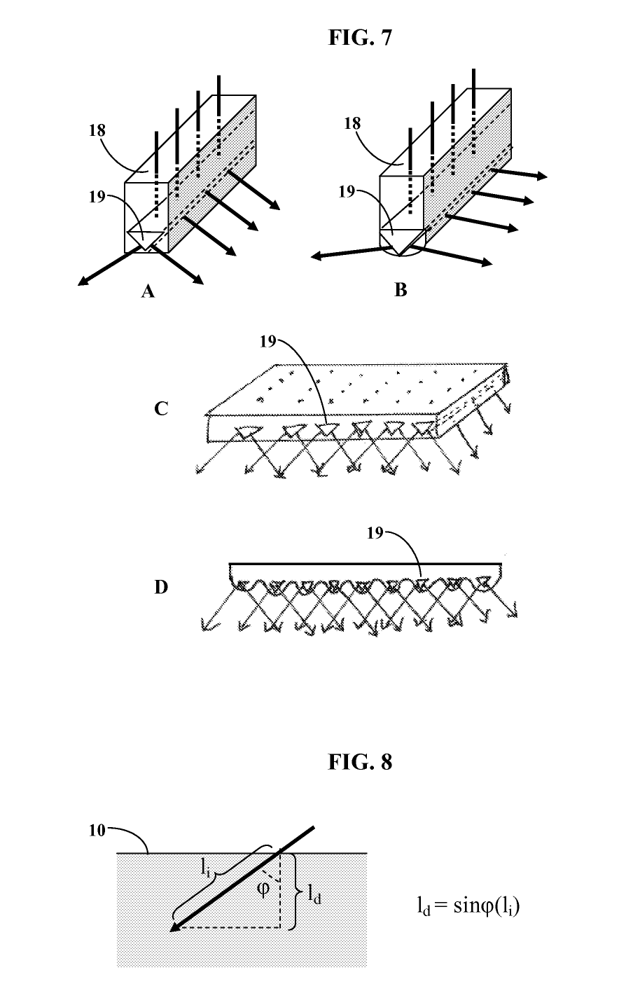

Incision units comprising a row are shown in FIG. 7, wherein FIG. 7A depicts a flat-headed incision unit and FIG. 7B depicts a rounded-head incision unit. Each of the incision units in FIG. 7 comprise a beam splitter 19 for partitioning a source laser into dual laser incisors. FIG. 7C depicts an incision unit featuring an array of flat-headed incision units, each of which includes a beam splitter 19 for partitioning a source laser into dual laser incisors, and FIG. 7D provides a side perspective view of an incision unit featuring an array of rounded-head incision units, each of which also includes a beam splitter 19 for partitioning a source laser into dual laser incisors.

Incision units comprising rows and arrays have certain advantages. First, rows and arrays are efficient because they have the capacity to deliver many “hits” and segment many follicles while keeping the cellular injury at a minimum and the spacing between insults to the body surface appropriate for rapid healing. Another advantage of the array configuration is that the face of such devices maintains the expected angle of the incisor (preferably, a laser) relative to the body surface. In this regard, one of the potential toxicities of such angled treatments is that the depth of skin injury is preferably kept to less than about 750 microns (for most applications) relative to the body surface. Therefore, in a single device head (FIG. 5) or a row configuration (FIGS. 7A and 7B), if the operator orients an incision unit at an angle that is other than perpendicular to the body surface, then the angled incisors might penetrate deeper than the well-tolerated depth of body surface injury. Consequently, with the array configuration, the large surface of the device head keeps the application face parallel to the body surface and provides a higher likelihood that the angled incisors do not injure tissue deeper than the prescribed amount. In order to reduce the possibility of improper angling of the incision unit, incision units may be configured with a large application face or a “cage” apparatus to ensure that the axis of the device is substantially perpendicular to the body surface such that the desired angle of the incisors are maintained.

FIG. 8 gives a trigonometric description of the laser angle φ, and the optimal length of injury (lᵢ) and the length of injury depth (l_{d}), such that l_{d}=(lᵢ sin φ, since the length of injury (lᵢ) is the hypotenuse of the triangle formed by angle φ and the injury depth (l_{d}) (which is perpendicular to the body surface.) In typical embodiments, l_{d} may be less than 750 microns, less than 600 microns, less than 500 microns, less than 400 microns, less than 300 microns, less than 250 microns, less than 200 microns, or less than 200 microns, whereas 1, may be about 15 mm, about 10 mm, about 8 mm, about 7 mm, about 5 mm, about 3 mm, about 2 mm, or less.

The present systems may be configured for translating an incisor relative to the body surface during the application of the incisor to the body surface. Thus, the present methods may further comprise translating an incisor relative to the body surface during the application of the incisor to the body surface or otherwise forming an incision having a length greater than its width in the body surface using the incisor. The translation of the incisor may be accomplished by translation of the incision unit that applies the incisor, by translation of the incisor itself, or both. The translation of the incision unit while an incisor is being applied to the body surface results in an injury to the body surface along the path of the translated incisor, thereby producing a slice- or slit-type injury (hereafter, a “slice”, which is effectively an incision having a length greater than its width) rather than an injury resembling a column or shaft. A slice that runs substantially parallel to the body surface has a greater likelihood of intersecting and segmenting a hair follicle than does a column that was drilled through the body surface essentially at a single point only. The efficiency of the present systems and methods (i.e., the ability to segment as many hair follicles as possible during a treatment session) is significantly improved through the novel process of translating the incisor relative to the body surface while the incisor is being applied thereto.

The incisor may be translated about 0.5 mm to about 5 mm relative to the body surface. For example, the incisor may be translated about 0.5 mm, about 1 mm, about 1.5 mm, about 2 mm, about 2.5 mm, about 3 mm, about 3.5 mm, about 4 mm, about 4.5 mm, or about 5 mm relative to the body surface. The “cage” of the incision unit (e.g., when the incision unit is configured for applying a fractional laser), or in the absence of a cage, any distal portion of an incision unit that is positioned proximate to the body surface, may comprise a mechanism for tracking the translation of the incisor. In other embodiments, some other component of the system (e.g., through an imager such as a camera or any other component that is separate from the incision unit) may be used to track the translation of the incisor, and may be configured to communicate with the incision unit either directly or indirectly (e.g., through a computer medium) so that the incisor may be activated or deactivated in response to translation, as appropriate. Fractional lasers traditionally include such tracking mechanisms to assess the velocity at which the laser unit is translated over the surface of, for example, skin, so that the individual points of the fractional laser pattern may be applied at the appropriate location, for the appropriate duration, and at the appropriate power. Tracking mechanisms may include mechanical rollers, lenses, laser-based tracking units, or any other appropriate mechanism. Those of ordinary skill in the art will readily appreciate these and other techniques for tracking the translation of the incisor relative to the body surface.

As provided above, translation of the incisor may be accomplished by translation of the incision unit that applies the incisor, by translation of the incisor itself, or both. The system is preferably configured to allow the incision unit to be moved in any direction relative to the body surface. For example, the incision unit may be associated with a movable element, such as an arm or other mounting or housing, that may be moved relative to the body surface under mechanized or manual (human) manipulation. The operation of the incision unit (e.g., its activation, deactivation, and movement thereof) may be under human, machine (e.g., computer), or mixed human and machine control. The components that may be necessary for moving a device such as the incision unit to any point on a two dimensional plane (corresponding to any point on the body surface), as well as any point in three dimensional space (and thereby any point in space relative to the body surface) are readily identified by those of ordinary skill in the art.

One or more incisors that are applied by the incision unit may be translated relative to the body surface during the application of such incisor(s). The incisor may be vibrated, shifted, or otherwise moved during application to the body surface in order to translate the incisor relative to the body surface. For example, those of ordinary skill in the art are familiar with mechanisms, such as piezoelectric elements, that are used to shift the location on which a point of fractional laser energy is directed, and any such mechanism may be used when the incisor is a fractional laser in order to translate the laser relative to the body surface while the laser is being applied thereto. Any other laser type or fluid jet may be translated relative to the body surface by moving the source of the laser beam or jetting nozzle, respectively, using the appropriate mechanism as will be readily appreciated among those skilled in the art. Where the incisor is a blade, needle, or another cutting implement, translation relative to the body surface may be provided by vibrating or otherwise moving the implement, preferably by precision controlled machinery in order to form an incision in the body surface that is of the appropriate length, depth, and orientation relative to the hair follicle.

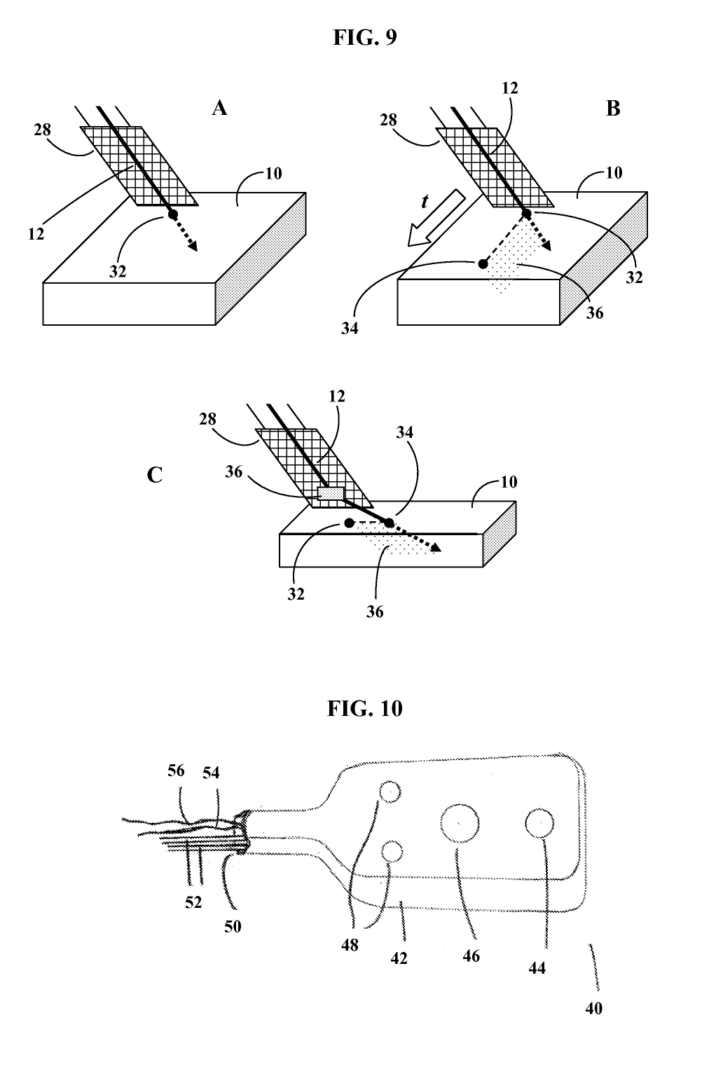

FIG. 9 depicts how translation of an incisor may be used to form a “slice” injury in a body surface. Any discussion in the present disclosure of a “channel” whereby a composition may be delivered to such injury is intended to apply to slice- or slit-type injuries as well. FIG. 9A shows an incision unit from which a laser incisor 12 is emitted and applied to body surface 10, whereby the incisor 12 enters the body surface 10 at point 32. Without translation of the incisor, a substantially column shaped injury will result from the application of incisor 12 to said body surface 10. In FIG. 9B, while incisor 12 is being applied to body surface 10, incision unit 28 is translated relative to body surface 10 in the transverse direction indicated by arrow t from the point 32 of initial entry into body surface 10 to endpoint 34, at which time incisor 12 is deactivated. The translation of incisor 12 from point 32 to point 34 leaves an incision 36 having a length corresponding to the distance from point 32 to point 34. In another embodiment, shown in FIG. 9C, while incisor 12 is being applied to body surface 10, piezoelectric element 36 redirects incisor 12 so that the incisor translated relative to body surface 10 from the point 32 of initial entry into body surface 10 to endpoint 34, at which time incisor 12 is deactivated. Incision unit 28 is not moved relative to body surface 10 in order to effect translation of incisor 12. The translation of incisor 12 from point 32 to point 34 using piezoelectric element 36 leaves an incision 36 having a length corresponding to the distance from point 32 to point 34.

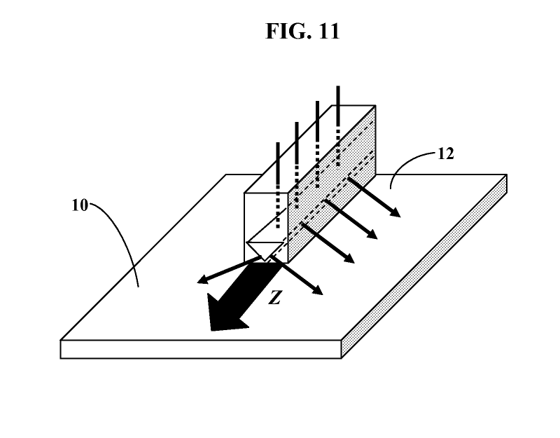

An incisor may also be translated relative to the body surface between the times during which the incisor is being applied to the body surface, i.e., when the incisor is not being applied to the body surface. Once a portion of the body surface has been subjected to treatment by application of an incisor for segmenting a hair follicle, the incisor, and preferably the incision unit may be translated to a further portion of the body surface for treatment thereof by application of the incisor to the further portion. The incision unit may be translated by association with a movable element, such as an arm or other mounting or housing, that may be moved relative to the body surface under mechanized or manual (human) manipulation. The translation of the incision unit relative to the body surface may be under human, machine (e.g., computer), or mixed human and machine control. As provided supra, the components that may be necessary for moving a device such as the incision unit to any point on a two dimensional plane (corresponding to any point on the body surface), as well as any point in three dimensional space (and thereby any point in space relative to the body surface) are readily identified by those of ordinary skill in the art. In some embodiments, the incision unit is translated from the first portion of the body surface to a further portion in a direction that is substantially transverse to the axis of the incisors that were applied to the first portion, although the translation of the incision unit may be in any desired direction. It may be desirable to translate the incision unit in accordance with the orientation of a population of hair follicles. For example, if a subject's scalp features a clockwise hair whorl, the incision unit may be translated relative to the scalp in a direction such as to follow the path of the whorl. FIG. 11 illustrates an embodiment whereby an incision unit having a “row” configuration is translated relative to the body surface 10 in a direction Z that is substantially transverse; the incisors are shown in order to illustrate the direction of translation, and would be deactivated during the translation of the incision unit from a first portion of the body surface to a further portion of the body surface. The translation of the incision unit from one portion of the body surface to a further portion of the body surface may be performed iteratively in order to effect treatment with respect to an entire body surface, as desired.

The present systems may further comprise an applicator for delivering a composition to the site of a hair follicle. The applicator may be any appropriate device for delivering compositions of the variety disclosed herein. The applicator may be configured for contacting the body surface with a composition by spraying, dripping, painting, propelling, misting, atomizing, or injecting, or may be configured for applying the composition by any combination of such methods. The application of the composition to the site of a hair follicle may be topical, may be to some location at the site of a hair follicle that is interior to the body surface, or both, and the applicator may be configured accordingly. In some embodiments, applicator is configured to deliver a composition that is a fluid onto the site of a hair follicle. Nozzles for dripping, misting, atomizing, or stream-spraying (e.g., in a flat or round stream) a fluid are well known in the art. The applicator may be configured for “painting” a composition onto the body surface, for example, as a brush, roller, or roller ball. Applicators for injecting a composition at the site of a hair follicle include needles, such as nano- or micro-injection needles. The applicator may be configured for applying a composition by iontophoresis, ultrasound penetration enhancement, electroporation, sponge application, or by any other suitable process. Preferably, the applicator is configured so that the delivery of the composition to the site of a hair follicle is spatially precise within a therapeutically acceptable margin of error. Exemplary devices for the propulsion of compositions comprising particles are disclosed in U.S. Pat. Nos. 6,306,119, 6,726,693, and 6,764,493, as well as WO 2009/061349.

The composition may comprise components that cause gelling or hardening of the composition (for example, the gelling or hardening may occur as a result of a reaction between two or more components within the composition), and the applicator may be configured for delivering a composition of this kind. To this end, the applicator may comprise a mixer for combining two or more gel-forming components prior to delivering the composition. The formation of the gel after the mixing of the gel-forming components may be delayed long enough for the composition to be delivered as fluid to the site of a hair follicle, or the gel may form substantially immediately after the mixing of the gel-forming components but either the gel may be capable of undergoing shear-thinning such that the gel may still be sprayed or otherwise delivered by the applicator, or the applicator may be configured for delivering a gel.

In other embodiments, the applicator may comprise components that substantially correspond to those used in inkjet technology. Thermal inkjets, piezoelectric inkjets, and continuous inkjets are the three main versions of this technology, and the components for the applicator may substantially correspond to those used in any of these types of inkjet systems. In other embodiments, the applicator may comprise components that substantially correspond to those used in inkjet technology. Thermal inkjets, piezoelectric inkjets, and continuous inkjets are the three main versions of this technology, and the components for the applicator may substantially correspond to those used in any of these types of inkjet systems. In such embodiments, the system may be configured to coordinate the activity of the incisor with that of the applicator. For example, the system may be configured to instruct the applicator to apply the composition to the precise spatial position of the site of incision; where the incisor removes a column of tissue at the site of the hair follicle to form a channel, the system may be configured to instruct the applicator to apply the composition into the channel. The system may be configured in this fashion through the use of computer software that determines the spatial position of the incisor at the time of injury and correlates this position to the precise site of injury and the location of the resulting channel, and then positions the applicator so that the composition is precisely directed into the channel using the inkjet technology. An imager may be used to assist in the determination of the location of the channel and the system may be configured to use this information in positioning or otherwise instructing the applicator.

The present systems may further comprise components that are capable of displacing or eliminating an impediment; such components may generally be referred to as “displacers”. As described above, the determination of the absence or presence of a physical feature may further comprise assessing the absence or presence of an impediment. A hair, a sweat droplet, oil, dirt, a mole, skin pigmentation, dead skin, a scab, or any combination thereof may be located at the body surface in such a manner as to constitute an impediment to assessment, treatment, or both. Even if the impediment does not interfere with assessment or treatment of the body surface, it may be desirable to avoid injuring the impediment. For example, if the impediment is a hair, it is typically desirable to avoid severing or otherwise damaging the hair, especially given an objective of the treatment is to promote hair growth or to increase the density of hair. Where an assessment is made that an impediment is present, it may be desirable to displace or eliminate the impediment, or to select a new location on the portion of the body surface for assessment. In other instances, it may not be necessary to address the presence of the impediment. A computer may be loaded with the appropriate software for identifying an impediment and determining if displacement or elimination of any such impediment is appropriate.

Depending on the type of impediment that is found, any of a variety of different approaches may be used to displace or eliminate the impediment. For example, forced air may be used to blow away, blow aside, or evaporate an impediment; a hair or a sweat droplet may be blown aside, dead skin or dirt may be blown away, and a sweat droplet may be evaporated. A stream of liquid, such as water, may also be used to displace an impediment. Devices for producing forced air (e.g., in a stream), a stream of liquid, or other suitable means for displacing or eliminating an impediment may be readily appreciated among those skilled in the art. Displacers may be separate from or integrated with any of the other components described herein with respect to the present systems. For example, the applicator or incisor may themselves be used to displace or eliminate an impediment; the applicator, incisor, or both may be equipped, for example, to deliver a stream of air or liquid. In other instances, the applicator and incisor are not themselves equipped to perform displacement or elimination of an impediment, but there may otherwise be a device associated with (e.g., occupying the same mounting as) either of these components that is capable of performing these tasks. In yet other embodiments, the displacer is separate from the applicator and incisor. Any method or device for displacing or eliminating an impediment may be used in accordance with the present disclosure.

The present systems may further comprise an imager for identifying the first hair follicle at the body surface prior to the application of the incisor. The “identification” of the first hair follicle by the imager may be in accordance with the preceding description with respect to the present methods. The imager may be any device that permits an assessment of the body surface to determine the location, angle, or any other relevant characteristic of a hair follicle. For example, a camera (e.g., a digital cameras, a charge-coupled device (CCD) camera, or the like) may be used to image a desired portion of the body surface. Other nonlimiting examples of imagers include any light- or sound-based system, such as a lens-bearing device (e.g., a microscope), a laser scanner, a sonar- or ultrasound-based device, a photoacoustic imager, or a fluoroscopic device. Preferably, imaging includes the acquisition of an image of the portion of the body surface at which a hair follicle is located and storage of the image, such as in electronic digital format. The present systems may further comprise suitable digital media for storing images. A stored image may then be used for subsequent assessments, including assessments of subparts of the image, such as the area equivalent to that which would be occupied by a further hair follicle and/or another physical feature, if present. The image is preferably acquired in sufficiently high resolution to locate and distinguish among hair follicles and other physical features.

The system may be configured for determining the absence or presence and location of one or more physical features. “Physical features” are discussed supra in connection with the disclosed methods and include hair follicles or indicia thereof. The system may be configured to allow a human operator to identify and locate a physical feature on the selected body surface, for example, by providing data that permits the human operator to determine whether a candidate physical feature is absent or present, and if present, where the physical feature is located on the body surface. The data that is provided to the human operator may be visual, acoustic, numerical, or any other relevant data that assists in the determination. In other embodiments, the system is configured to obtain data from the body surface that permits the system to make the determination without or substantially without human intervention. The system may be equipped with software that assesses the characteristics of a physical feature in order to perform an identification, that distinguishes between different physical features, that determines the location of a physical feature on a portion of the body surface (e.g., relative to other physical features, to the margins of the portion of the surface, or both). The assessment may include a hierarchy of decisions that are binary (e.g., “hair related” or “not hair related”), involve a choice from among multiple options (“hair” or “vellus hair” or “hair pore”, and the like), or both. The determination of the absence or presence and location of one or more physical features may in turn be used by the system to determine whether or not an incisor should be applied, how the incisor or incision unit should be positioned, whether a composition should be applied by the applicator, or to make other pertinent decisions.

The components of the present systems may be substantially separate or may be integrated into a unitized structure. Any subset of the system components may be integrated (e.g., an incision unit and an applicator), or all of the components may be substantially separate.

In accordance with some embodiments of this invention, unitary or cooperative devices may be employed to achieve desired action upon such surfaces. Thus, a plurality of functions may be integrated into a single ‘head’ or into a plurality of ‘heads’ which cooperate with each other, such as under control of a computer or operator, to achieve desired actions upon the surfaces. The functions which may be integrated include, among other things, imaging, injuring, and composition applying. For some embodiments, imaging, especially by camera, injuring, such as with a laser and composition applying, such as by ‘ink jetting’ techniques are integrated together into a single ‘head.’ The apparatuses may be effectively miniaturized such that the working head carrying them may be introduced into the corpus of a subject through arterial access. Larger heads may be used where convenient for the intended uses.

The composition delivery orifices may deliver a large variety of liquids including water, aqueous therapeutic solutions or slurries, liquid pharmaceuticals, dyes, indicators, radioopacifiers, radiotherapeutic absorptive materials, such as for subsequent application of radiofrequency, magnetic or other energy, or other things, as provided supra and appreciated among those having ordinary skill in the art. Such liquids may include liposomes, polymersomes, nanoparticles or other things for the delivery of drugs or therapeutic agents to specific locales. In one embodiment, one or more dispensing orifices are disposed to as to rinse or clean the imaging device or lens, the injuring, e.g., laser, portion of the head or other things so as to provide a clear field of view and unimpeded field of action for the devices comprising the head.

An example of one integrated head is shown in FIG. 10. An integrated head 40 comprises a body 44, which may be conveniently molded to include locations for placement of apparatuses for accomplishing the desired actions. Thus, an imager, such as a camera 44 is included together with an incision unit, such as a, preferred, laser 46. These may also be integrated in the head 42 with one or more fluid composition deliver orifices 48, such as “ink jets.” Control, power, sensing, fluid providing and other feeds are also provided to the internal area of the head 50, including, for example, fluid supplies 52, power supplies 54 and control circuitry 56. Several of each of these may be included as needed to effect control, powering, and materials supply to the head.

In other embodiments, a plurality of integrated (or non-integrated, but cooperative) heads may be provided to accomplish action upon surfaces. In other embodiments, one or more heads are operated under computer or robotic control. Placement apparatus, such as an X-Y positioner, many examples of which are known per se, may be used to move the head under operational control, to specific locations. Such positioners may be controlled manually by an operator, or the same may be controlled by a computer or robotic controller. Each of these is also known per se and such control is well within the skill of routineers in the art. It is particularly preferred, when employing a positioner for the head or heads, to provide common control between the head and the positioner to enable action at a selected surface location to cooperate with positioning of the head at that location. Serial positioning and action accomplishment may be attained thereby and will accord convenience and efficacious action.

In certain embodiments, at least one of the incision unit, displacer, imager, and applicator may be under the operative control of a general purpose digital computer. In some embodiments, two of the incision unit, displacer, imager, and applicator are under the operative control of the general purpose digital computer, and in other embodiments, all of the incision unit, displacer, imager, and applicator are under the operative control of a computer.

Where any of the imager, incision unit, applicator, and displacer are under the operative control of a general purpose digital computer, the computer may be configured to enable the components thereof to operate in a substantially coordinated fashion; to select a location on the body surface having a preselected geometry with respect to a hair follicle; to assess and optionally adjust the location of a further target area in response to imaging; to instruct the incision unit to apply an incisor to the location of the further target area in response the assessment; to perform the assessment and optional adjustment iteratively to give rise to one or more additional target areas; or any combination thereof. In certain embodiments, the imager, incision unit, and applicator are all operatively linked via general purpose digital computer.

The system may be configured to enable any pair or all of the components thereof to operate in a substantially coordinated fashion. The computer may control such aspects as the activation and deactivation of the components relative to one another; the determination of the manner in which the incision unit applies one or more incisors based on the determination of the absence or presence of a particular type of physical feature that is present at a target area; or other actions that require coordination between or among components.

### Example 1

### Method of Promoting Hair Growth

A method according to the present invention for promoting new hair growth a human scalp is performed as follows. A male subject with substantial hair loss on the scalp is seated in a stationary examination chair. A high-resolution digital camera is used to obtain an image of an area of the scalp measuring about 100 cm². The image is stored onto the hard drive of a general purpose digital computer that is equipped with software for identifying physical features that are typically found on the scalp and for assigning coordinates to identified physical features that are based on the location of the physical feature relative to the margins of the imaged portion of the scalp. The computer identifies a first hair follicle by location of the point at which a detected terminal hair enters the scalp and records the likely location of the identified hair follicle, as well as the probable angle of the follicle in view of the angle of the terminal hair that is associated therewith.

An incision unit comprising an 5×5 array of fractional lasers is positioned so that one of the lasers is proximate to the identified hair follicle. Each unit of the 5×5 array is equipped with a laser for producing a fractional laser pattern, an angled “cage” that rests against the body surface in order to angle the laser so that it is applied to the body surface at an oblique angle when activated, and piezoelectric elements for directing individual beams. The system of which the incision unit is a part is controlled by a general purpose digital computer that accepts input regarding pertinent information from a human operator. The computer activates the incision unit, and each fractional laser produces a pattern of angled laser beams that are applied to the body surface. The computer controls the power of the lasers and the amount of time during which the laser is applied to the body surface so that each laser penetrates the body surface to a depth of 500 microns. The computer likewise controls the piezoelectric elements with respect to each beam in order to translate each beam relative to the body surface in a linear direction during application to the bodys surface, such that each beam produces a slit-like injury measuring 2 mm in length (and 500 microns deep, as specified above). The lasers are subsequently deactivated.

Next, the computer activates applicators that are integrated with the incision unit. The applicators each include an inkjet-type head for delivering a composition substantially directly into the wounds that were formed by the respective fractional beams. A small volume (about 50 μL) of a composition comprising 6-bromo-indirubin-3′-oxime (a GSK3β modulator) and carrier comprising PEO-PPO-PEO (a thermoreversible polymer that gels when exposed to human physiological temperatures) is delivered as a fluid into every third wound, and the applicators are deactivated.

The computer then uses the previously acquired image of the portion of the subject's scalp to permit a determination of the optimal direction in which the incision unit should be translated relative to the scalp. A human operator analyzes the image and determines that the remaining hairs on the subject's scalp are oriented in a clockwise whorl. The operator designates a direction in which the incision unit should be translated relative to the scalp in order to expose as many follicles at an appropriate angle to the incisors, and enters the appropriate information to the computer using an interface. The computer then directs the incision unit to a location relative to the subject's scalp that is consistent with the determination of the orientation of hair follicles. The computer then activates the incision unit, and each fractional laser produces a pattern of angled laser beams that are applied to the body surface. The computer controls the power of the lasers and the amount of time during which the laser is applied to the body surface so that each laser penetrates the body surface to a depth of 500 microns. The computer likewise controls the piezoelectric elements with respect to each beam in order to translate each beam relative to the body surface in a linear direction during application to the bodys surface, such that each beam produces a slit-like injury measuring 2 mm in length (and 500 microns deep, as specified above). The lasers are subsequently deactivated.

The described process is performed iteratively to give rise to additional treatments of the body surface until substantially all of the subject's scalp has been subjected to angled laser treatment.

What is claimed:

1. A method for stimulating hair growth at a body surface comprising:
(a) identifying a first hair follicle on said body surface;
(b) segmenting said first hair follicle into at least two disunited subunits in response to said identification;
(c) optionally applying a composition to the site of said first hair follicle; and,
(d) identifying a further hair follicle;
segmenting said further hair follicle into at least two disunited subunits; and
optionally applying the same or a different composition to the site of said further hair follicle;
Or,
(e) segmenting each of one or more further hair follicles into at least two disunited subunits contemporaneously with step (b); and
optionally applying the same or a different composition to the site of said further hair follicle contemporaneously with step (c).

2. The method of claim 1 wherein step (d) is performed iteratively to segment one or more additional hair follicles.

3. The method according to claim 1 further comprising assessing the absence or presence of an impediment at the site of said hair follicle; and
optionally displacing said impediment in response to said assessment, or
selecting a location on the body surface having a preselected geometry with respect to the first hair follicle.

4. The method according to claim 3 wherein said impediment is a hair, a sweat droplet, oil, dirt, a mole, skin pigmentation, or any combination thereof

5. The method according to claim 1 wherein said first hair follicle, said further hair follicle, or both are segmented by applying an incisor at an oblique angle relative to said body surface at the respective locations of said follicles.

6. The method according to claim 5 wherein said incisor is applied at an angle of about 85° or less relative to said body surface.

7. The method according to claim 5 wherein said incisor is translated relative to said body surface during the application thereof to said body surface.

8. The method according to claim 7 wherein said incisor is translated about 0.5 mm to about 5 mm relative to said body surface during the application thereof to said body surface.

9. The method according to claim 5 wherein said incisor is applied at an angle φ relative to axis y that is perpendicular to said body surface, wherein said first hair follicle, said further hair follicle, or both are independently oriented at an angle α relative to said body surface, wherein the sum of angle α and an angle β is 90°, and wherein the sum of angle φ and angle φ is about 65° to about 115°.

10. The method according to claim 9 wherein the sum of angle φ and angle β is about 90°.

11. The method according to claim 5 wherein said incisor is a laser.

12. The method of claim 5 wherein said first hair follicle, said further hair follicle, or both are segmented by removing a column of tissue to form a channel that transects said first hair follicle, said further hair follicle, or both.

13. The method according to claim 1 wherein said composition comprises a fluid.

14. A system for stimulating hair growth at a body surface comprising:
an incision unit that is configured for applying a first incisor at an oblique angle relative to said body surface at the location of said first hair follicle for segmenting said first hair follicle into at least two disunited subunits.

15. The system according to claim 14 wherein said system is configured for translating said incisor relative to said body surface during the application of said incisor to said body surface.

16. The system according to claim 15 wherein said system is configured for translating said incisor about 0.5 mm to about 5 mm relative to said body surface during the application of said incisor to said body surface.

17. The system according to claim 14 wherein said incisor is applied at an angle of about 85° or less relative to said body surface.

18. The system according to claim 14 wherein said incision unit is configured for:
partitioning a source laser beam into at least a first laser incisor and a second laser incisor,
applying said first laser incisor to said body surface at an oblique angle relative to said body surface; and
applying said second laser incisor at an oblique angle relative to said body surface,
wherein said first laser incisor and said second laser incisor are applied to said body surface contemporaneously.

19. The system according to 18 wherein said first laser incisor, said second laser incisor, or both are each applied at an angle of about 85° or less relative to said body surface.

20. The system according to claim 18 wherein said laser is a fractional laser.

21. The system according to claim 18 wherein said incision unit is configured for
partitioning each of at least two source laser beams into respective first and second laser incisors;
applying each of said first and second laser incisors to said body surface at an oblique angle relative to said body surface.

22. The system according to claim 21 wherein each of said first and second laser incisors are applied to said body surface at an angle of about 85° or less relative to said body surface.

23. The system according to claim 18 wherein said system is configured for translating said first and second laser incisors relative to said body surface during the application of said first and second laser incisors to said body surface.

24. The system according to claim 23 wherein said system is configured for translating said first and second laser incisors about 0.5 mm to about 5 mm relative to said body surface during the application of said first and second laser incisors to said body surface.

25. The system according to claim 14 further comprising an imager for identifying said first hair follicle at said body surface prior to application of said incisor.

26. The system according to claim 14 further comprising an applicator for delivering a physiologically active composition to the site of said first hair follicle.

27. The system according to claim 26 further comprising an imager for identifying said first hair follicle at said body surface prior to application of said incisor.

28. The system according to claim 27 wherein at least two of the imager, applicator, and incision unit are under the operative control of a general purpose digital computer.

29. The system according to claim 28 wherein said computer is configured for selecting a location on the body surface having a preselected geometry with respect to the first hair follicle.

30. The system according to claim 29 wherein said computer is configured for assessing and optionally adjusting said location.

31. The system according to claim 30 wherein said computer is configured for instructing said incision unit to apply said first incisor to said location in response to said assessment.

32. The system according to claim 26 wherein said applicator is configured for delivering a fluid.

33. The system according to claim 32 wherein said applicator further comprises a mixer for combining two or more gel-forming components and a physiologically active ingredient prior to delivering said composition.

34. The system according to claim 32 wherein said applicator comprises components that substantially correspond to those used in inkjet technology.
